(19) United States
(12) Patent Application Publication
(10) Pub. No.: US 2022/0262463 A1
(43) Pub. Date: Aug. 18, 2022

(54) METHOD FOR SIMULATING OLIGOMER OR POLYMER GROWTH

(71) Applicant: COVESTRO LLC, Pittsburgh, PA (US)

(72) Inventor: James A THOMPSON-COLÓN, Moon Township, PA (US)

(21) Appl. No.: 17/641,909

(22) PCT Filed: Sep. 16, 2020
(86) PCT No.: PCT/US2020/050961

§ 371 Date: Mar. 10, 2022

Related U.S. Application Data
(60) Provisional application No. 62/903,153, filed on Sep. 20, 2019.

(51) Int. Cl. G16C 20/10; G16C 20/40; G16C 20/70

(52) U.S. Cl. CPC G16C 20/10; G16C 20/70; G16C 20/40

(57) ABSTRACT

A method for simulating oligomer or polymer growth includes: receiving a reaction recipe including a plurality of reactive molecules; for each reactive molecule of the plurality of reactive molecules, determining at least one functional group associated with the reactive molecule; assigning a functional group type to each of the functional groups associated with the plurality of reactive molecules; determining at least one reaction rule associated with each functional group type; simulating a plurality of oligomer or polymer forming reactions from the plurality of reactive molecules based on the at least one reaction rule to form a plurality of simulated oligomers or polymers; and determining at least one oligomer or polymer structure associated with a first oligomer or polymer of the plurality of simulated oligomers or polymers.

### BACKGROUND

### 1. Field

The present disclosure relates to simulating oligomer or polymer growth and, in some non-limiting embodiments aspects, to methods, systems, and computer program products for simulating oligomer or polymer growth.

### 2. Technical Considerations

Understanding the resulting structure of an oligomer or polymer based on a set of reactants requires significant amounts of laboratory experimentation, which requires the expenditure of significant time and financial resources. Further, determining the performance properties associated with the formed oligomers or polymers requires further testing, resulting in additional expenditure of time and resources. Not all of these experiments result in oligomers or polymers with useful structures and/or performance properties, meaning at least some of the experimentation and testing is for naught.

### SUMMARY

According to some non-limiting embodiments or aspects, a method for simulating oligomer or polymer growth includes: receiving, with at least one processor, a reaction recipe including a plurality of reactive molecules; for each reactive molecule of the plurality of reactive molecules, determining, with at least one processor, at least one functional group associated with the reactive molecule; assigning, with at least one processor, a functional group type to each of the functional groups associated with the plurality of reactive molecules; determining, with at least one processor, at least one reaction rule associated with each functional group type; simulating, with at least one processor, a plurality of oligomer or polymer forming reactions from the plurality of reactive molecules based on the at least one reaction rule to form a plurality of simulated oligomers or polymers; and determining, with at least one processor, at least one oligomer or polymer structure associated with a first oligomer or polymer of the plurality of simulated oligomers or polymers.

In some non-limiting embodiments or aspects, simulating the plurality of oligomer or polymer forming reactions may include: associating, with at least one processor, at least one functional group associated with the plurality of reactive molecules with at least one other functional group associated with the plurality of reactive molecules. Simulating the plurality of oligomer or polymer forming reactions may include: generating, with at least one processor, a first list of a plurality of first type reactive functional groups of the reactive functional groups associated with the plurality of reactive molecules and a second list of a plurality of second type reactive functional groups of the reactive functional groups associated with the plurality of reactive molecules; randomizing, with at least one processor, an order of the plurality of first type reactive functional groups in the first list; randomizing, with at least one processor, an order of the plurality of second type reactive functional groups in the second list; associating, with at least one processor, at least one of the first type reactive functional groups from the first list with at least one of the corresponding second reactive functional groups from the second list based on the randomized orders to form at least one simulated bond of the first oligomer or polymer. The method may include generating, with at least one processor, statistical reaction data based on the plurality of oligomer or polymer forming reactions. Simulating the plurality of oligomer or polymer forming reactions may include: associating, with at least one processor, at least one pair of the functional groups associated with the plurality of reactive molecules based on the at least one reaction rule to form a bonded pair. The method may include assigning, with at least one processor, a bonded group identifier associated with the bonded pair. Simulating the plurality of oligomer or polymer forming reactions may include: associating, with at least one processor, a first pair of functional groups associated with the plurality of reactive molecules based on the at least one reaction rule to form a first bonded pair; and subsequently associating, with at least one processor, a second pair of functional groups associated with the plurality of reactive molecules based on the at least one reaction rule to form a second bonded pair. Simulating the plurality of oligomer or polymer forming reactions may further include: adjusting, with at least one processor, the at least one reaction rule between associating the first pair of functional groups and associating the second pair of functional groups. The method may further include: determining, with at least one processor, at least one characteristic associated with the first oligomer or polymer. The at least one characteristic may include at least one of the following: moles of effective links per kg of oligomer or polymer, moles of effective links per kg of gel component, moles of effective links per kg of core in a gel component, moles of intramolecular rings formed per kg of oligomer or polymer, moles of intermolecular rings formed per kg of gel component, moles of intermolecular rings formed per kg of core of a gel component, crosslink density, moles of crosslink junctions per kg of oligomer or polymer, moles of dangler links per kg of oligomer or polymer, moles of danglers per kg of gel component, percent weight of sol in gelled oligomer or polymer, percent weight of gel in gelled oligomer or polymer, percent weight of danglers, percent weight of core gel, number average molecular weight of elastic links, weight average molecular weight of elastic links, number average molecular weight of danglers, weight average molecular weight of danglers, molecular weight of danglers weighted by its percent of total oligomer or polymer, molecular weight of elastic links weighted by its percent weight of the oligomer or polymer, number average molecular weight, weight average molecular weight, z-average molecular weight, degree of polymerization, dispersity of reaction product, number of ingredient molecules used in simulation, number of monomers used in simulation, number of ring closures formed, number of oligomer molecules formed, ratio of equivalents (CO/OH) of raw materials, number average OH functionality, number average CO functionality, functional-average functionality of CO, weight average functionality of OH, weight-average OH functionality, weight-average CO functionality, average new bonds formed per oligomer, moles of bonds formed per kg of oligomer or polymer, moles of remaining OH groups per kg of oligomer or polymer, moles of remaining CO groups per kg of oligomer or polymer, OH number, acid number if CO is a carboxylic acid, percent weight of isocyanate group in products, percent isocyanate in product stripped of any isocyanate monomer, extent of reaction, percent weight of unreacted monomers, number average molecular weight of hard segment, average number of monomers per hard segment, molecular weight of danglers connected to hard segment, and average number of monomers per soft segment. The method may further include: determining, with at least one processor, at least one expected property associated with the first oligomer or polymer based on the determined at least one characteristic associated with the first oligomer or polymer. The at least one expected property may include at least one of the following: a mechanical testing property, a physical testing property, a thermal testing property, a rheological testing property, a barrier testing property, a weathering and/or chemical resistance testing property, an adhesion testing property, a flammability testing property, an optical testing property, and an electrical testing property.

In some non-limiting embodiments or aspects, simulating the plurality of oligomer or polymer forming reactions may include: determining, with at least one processor, an extent of reaction associated with the plurality of simulated oligomer or polymer forming reactions. The at least one reaction rule may include a relative reactivity of at least one functional group type. The at least one reaction rule may include a first functional group type capable of undergoing a reaction with a second functional group type. The reaction recipe may include an initial plurality of reactive molecules and a subsequent plurality of reactive molecules, where simulating the plurality of oligomer or polymer forming reactions may include simulating the oligomer or polymer forming reactions from the initial plurality of reactive molecules based on the at least one reaction rule, where the method may further include simulating, with at least one processor, a plurality of subsequent oligomer or polymer forming reactions from the plurality of subsequent reactive molecules and molecules and/or oligomers and/or polymers formed from the plurality of oligomer or polymer forming reactions based on the at least one reaction rule. The method may further include: generating, with at least one processor, reaction instructions for forming the first oligomer or polymer. The method may further include: communicating, with at least one processor, the reaction instructions to a reactor to cause the reactor to initiate preparation of the first oligomer or polymer. Determining the at least one oligomer or polymer structure associated with the first oligomer or polymer may include determining simulated danglers, sols, and elastic links. Determining the at least one expected property may include analyzing the at least one characteristic based on historical data associated with oligomers or polymers. Analyzing the at least one characteristic based on the historical data may include generating the at least one expected property using a machine learning algorithm. The method may further include: storing, with at least one processor, historical simulation data associated with the plurality of simulated oligomers or polymers; receiving, with at least one processor, a recommendation request, where the recommendation request may include at least one target physical property associated with an oligomer or polymer desired to be produced; querying, with at least one processor, the stored historical simulation data; and generating, with at least one processor, a recommendation response including reaction instructions for forming an oligomer or polymer having the at least one target physical property based on the historical simulation data. Determining the at least one oligomer or polymer structure associated with the first oligomer or polymer may include identifying the at least one oligomer or polymer structure associated with the first oligomer or polymer based on a component searching algorithm.

In some non-limiting embodiments or aspects, the first oligomer or polymer may include a cured thermoset oligomer or polymer. Determining the at least one oligomer or polymer structure associated with the first oligomer or polymer may include at least one of the following: identifying, with at least one processor, a soft segment of the first oligomer or polymer and/or a hard segment of the first oligomer or polymer; and analyzing, with at least one processor, the soft segment of the first oligomer or polymer and/or the hard segment of the first oligomer or polymer. The method may further include: storing, with at least one processor, historical simulation data associated with the plurality of simulated oligomers or polymers; receiving, with at least one processor, a message from a reactor, the message including at least one property associated with a material being prepared in the reactor; determining, with at least one processor, at least one reactor adjustment based on the message and the historical simulation data; and communicating, with at least one processor, a reply message to the reactor to cause the reactor to initiate the reactor adjustment.

According to some non-limiting embodiments or aspects, a system for simulating oligomer or polymer growth may include at least one processor programmed or configured to: receive a reaction recipe including a plurality of reactive molecules; for each reactive molecule of the plurality of reactive molecules, determine at least one functional group associated with the reactive molecule; assign a functional group type to each of the functional groups associated with the plurality of reactive molecules; determine at least one reaction rule associated with each functional group type; simulate a plurality of oligomer or polymer forming reactions from the plurality of reactive molecules based on the at least one reaction rule to form a plurality of simulated oligomers or polymers; and determine at least one oligomer or polymer structure associated with a first oligomer or polymer of the plurality of simulated oligomers or polymers.

In some non-limiting embodiments or aspects, simulating the plurality of oligomer or polymer forming reactions may include the at least one processor being programmed or configured to: associate at least one functional group associated with the plurality of reactive molecules with at least one other functional group associated with the plurality of reactive molecules. Simulating the plurality of oligomer or polymer forming reactions may include the at least one processor being programmed or configured to: generate a first list of a plurality of first type reactive functional groups of the reactive functional groups associated with the plurality of reactive molecules and a second list of a plurality of second type reactive functional groups of the reactive functional groups associated with the plurality of reactive molecules; randomize an order of the plurality of first type reactive functional groups in the first list; randomize an order of the plurality of second type reactive functional groups in the second list; associate at least one of the first type reactive functional groups from the first list with at least one of the corresponding second reactive functional groups from the second list based on the randomized orders to form at least one simulated bond of the first oligomer or polymer. The at least one processor may be programmed or configured to: generate statistical reaction data based on the plurality of oligomer or polymer forming reactions. Simulating the plurality of oligomer or polymer forming reactions may include the at least one processor being programmed or configured to: associate at least one pair of the functional groups associated with the plurality of reactive molecules based on the at least one reaction rule to form a bonded pair. The at least one processor may be programmed or configured to: assign a bonded group identifier associated with the bonded pair. Simulating the plurality of oligomer or polymer forming reactions may include the at least one processor being programmed or configured to: associate a first pair of functional groups associated with the plurality of reactive molecules based on the at least one reaction rule to form a first bonded pair; and subsequently associate a second pair of functional groups associated with the plurality of reactive molecules based on the at least one reaction rule to form a second bonded pair. Simulating the plurality of oligomer or polymer forming reactions may include the at least one processor being programmed or configured to: adjust the at least one reaction rule between associating the first pair of functional groups and associating the second pair of functional groups. The at least one processor may be programmed or configured to: determine at least one characteristic associated with the first oligomer or polymer. The at least one characteristic may include at least one of the following: moles of effective links per kg of oligomer or polymer, moles of effective links per kg of gel component, moles of effective links per kg of core in a gel component, moles of intramolecular rings formed per kg of oligomer or polymer, moles of intermolecular rings formed per kg of gel component, moles of intermolecular rings formed per kg of core of a gel component, crosslink density, moles of crosslink junctions per kg of oligomer or polymer, moles of dangler links per kg of oligomer or polymer, moles of danglers per kg of gel component, percent weight of sol in gelled oligomer or polymer, percent weight of gel in gelled oligomer or polymer, percent weight of danglers, percent weight of core gel, number average molecular weight of elastic links, weight average molecular weight of elastic links, number average molecular weight of danglers, weight average molecular weight of danglers, molecular weight of danglers weighted by its percent of total oligomer or polymer, molecular weight of elastic links weighted by its percent weight of the oligomer or polymer, number average molecular weight, weight average molecular weight, z-average molecular weight, degree of polymerization, dispersity of reaction product, number of ingredient molecules used in simulation, number of monomers used in simulation, number of ring closures formed, number of oligomer molecules formed, ratio of equivalents (CO/OH) of raw materials, number average OH functionality, number average CO functionality, functional-average functionality of CO, weight average functionality of OH, weight-average OH functionality, weight-average CO functionality, average new bonds formed per oligomer, moles of bonds formed per kg of oligomer or polymer, moles of remaining OH groups per kg of oligomer or polymer, moles of remaining CO groups per kg of oligomer or polymer, OH number, acid number if CO is a carboxylic acid, percent weight of isocyanate group in products, percent isocyanate in product stripped of any isocyanate monomer, extent of reaction, percent weight of unreacted monomers, number average molecular weight of hard segment, average number of monomers per hard segment, molecular weight of danglers connected to hard segment, and average number of monomers per soft segment. The at least one processor may be programmed or configured to: determine at least one expected property associated with the first oligomer or polymer based on the determined at least one characteristic associated with the first oligomer or polymer. The at least one expected property may include at least one of the following: a mechanical testing property, a physical testing property, a thermal testing property, a rheological testing property, a barrier testing property, a weathering and/or chemical resistance testing property, an adhesion testing property, a flammability testing property, an optical testing property, and an electrical testing property.

In some non-limiting embodiments or aspects, simulating the plurality of oligomer or polymer forming reactions may include the at least one processor being programmed or configured to: determine an extent of reaction associated with the plurality of simulated oligomer or polymer forming reactions. The at least one reaction rule may include a relative reactivity of at least one functional group type. The at least one reaction rule may include a first functional group type capable of undergoing a reaction with a second functional group type. The reaction recipe may include an initial plurality of reactive molecules and a subsequent plurality of reactive molecules, where simulating the plurality of oligomer or polymer forming reactions may include simulating the oligomer or polymer forming reactions from the initial plurality of reactive molecules based on the at least one reaction rule, where the at least one processor is programmed or configured to simulate a plurality of subsequent oligomer or polymer forming reactions from the plurality of subsequent reactive molecules and molecules and/or oligomers and/or polymers formed from the plurality of oligomer or polymer forming reactions based on the at least one reaction rule. The at least one processor may be programmed or configured to: generate reaction instructions for forming the first oligomer or polymer. The at least one processor may be programmed or configured to: communicate the reaction instructions to a reactor to cause the reactor to initiate preparation of the first oligomer or polymer. Determining the at least one oligomer or polymer structure associated with the first oligomer or polymer may include determining simulated danglers, sols, and elastic links. Determining the at least one expected property may include analyzing the at least one characteristic based on historical data associated with oligomers or polymers. Analyzing the at least one characteristic based on the historical data may include generating the at least one expected property using a machine learning algorithm. The at least one processor may be programmed or configured to: store historical simulation data associated with the plurality of simulated oligomers or polymers; receive a recommendation request, where the recommendation request may include at least one target physical property associated with an oligomer or polymer desired to be produced; query the stored historical simulation data; and generate a recommendation response including reaction instructions for forming an oligomer or polymer having the at least one target physical property based on the historical simulation data. Determining the at least one oligomer or polymer structure associated with the first oligomer or polymer may include identifying the at least one oligomer or polymer structure associated with the first oligomer or polymer based on a component searching algorithm.

In some non-limiting embodiments or aspects, the first oligomer or polymer may include a cured thermoset oligomer or polymer. Determining the at least one oligomer or polymer structure associated with the first oligomer or polymer may include the at least one processor programmed or configured to: identify a soft segment of the first oligomer or polymer and/or a hard segment of the first oligomer or polymer; and analyze the soft segment of the first oligomer or polymer and/or the hard segment of the first oligomer or polymer. The at least one processor may be programmed or configured to: store historical simulation data associated with the plurality of simulated oligomers or polymers; receive a message from a reactor, the message including at least one property associated with a material being prepared in the reactor; determine at least one reactor adjustment based on the message and the historical simulation data; and communicate a reply message to the reactor to cause the reactor to initiate the reactor adjustment.

According to some non-limiting embodiments or aspects, a computer program product for simulating oligomer or polymer growth includes at least one non-transitory computer-readable medium including one or more instructions that, when executed by at least one processor, cause the at least one processor to: receive a reaction recipe including a plurality of reactive molecules; for each reactive molecule of the plurality of reactive molecules, determine at least one functional group associated with the reactive molecule; assign a functional group type to each of the functional groups associated with the plurality of reactive molecules; determine at least one reaction rule associated with each functional group type; simulate a plurality of oligomer or polymer forming reactions from the plurality of reactive molecules based on the at least one reaction rule to form a plurality of simulated oligomers or polymers; and determine at least one oligomer or polymer structure associated with a first oligomer or polymer of the plurality of simulated oligomers or polymers.

In some non-limiting embodiments or aspects, simulating the plurality of oligomer or polymer forming reactions may include the one or more instructions causing the at least one processor to: associate at least one functional group associated with the plurality of reactive molecules with at least one other functional group associated with the plurality of reactive molecules. Simulating the plurality of oligomer or polymer forming reactions may include the one or more instructions causing the at least one processor to: generate a first list of a plurality of first type reactive functional groups of the reactive functional groups associated with the plurality of reactive molecules and a second list of a plurality of second type reactive functional groups of the reactive functional groups associated with the plurality of reactive molecules; randomize an order of the plurality of first type reactive functional groups in the first list; randomize an order of the plurality of second type reactive functional groups in the second list; associate at least one of the first type reactive functional groups from the first list with at least one of the corresponding second reactive functional groups from the second list based on the randomized orders to form at least one simulated bond of the first oligomer or polymer. The one or more instructions may cause the at least one processor to: generate statistical reaction data based on the plurality of oligomer or polymer forming reactions. Simulating the plurality of oligomer or polymer forming reactions may include the one or more instructions causing the at least one processor to: associate at least one pair of the functional groups associated with the plurality of reactive molecules based on the at least one reaction rule to form a bonded pair. The one or more instructions may cause the at least one processor to: assign a bonded group identifier associated with the bonded pair. Simulating the plurality of oligomer or polymer forming reactions may include the one or more instructions causing the at least one processor to: associate a first pair of functional groups associated with the plurality of reactive molecules based on the at least one reaction rule to form a first bonded pair; and subsequently associate a second pair of functional groups associated with the plurality of reactive molecules based on the at least one reaction rule to form a second bonded pair. Simulating the plurality of oligomer or polymer forming reactions may include the one or more instructions causing the at least one processor to: adjust the at least one reaction rule between associating the first pair of functional groups and associating the second pair of functional groups. The one or more instructions may cause the at least one processor to: determine at least one characteristic associated with the first oligomer or polymer. The at least one characteristic may include at least one of the following: moles of effective links per kg of oligomer or polymer, moles of effective links per kg of gel component, moles of effective links per kg of core in a gel component, moles of intramolecular rings formed per kg of oligomer or polymer, moles of intermolecular rings formed per kg of gel component, moles of intermolecular rings formed per kg of core of a gel component, crosslink density, moles of crosslink junctions per kg of oligomer or polymer, moles of dangler links per kg of oligomer or polymer, moles of danglers per kg of gel component, percent weight of sol in gelled oligomer or polymer, percent weight of gel in gelled oligomer or polymer, percent weight of danglers, percent weight of core gel, number average molecular weight of elastic links, weight average molecular weight of elastic links, number average molecular weight of danglers, weight average molecular weight of danglers, molecular weight of danglers weighted by its percent of total oligomer or polymer, molecular weight of elastic links weighted by its percent weight of the oligomer or polymer, number average molecular weight, weight average molecular weight, z-average molecular weight, degree of polymerization, dispersity of reaction product, number of ingredient molecules used in simulation, number of monomers used in simulation, number of ring closures formed, number of oligomer molecules formed, ratio of equivalents (CO/OH) of raw materials, number average OH functionality, number average CO functionality, functional-average functionality of CO, weight average functionality of OH, weight-average OH functionality, weight-average CO functionality, average new bonds formed per oligomer, moles of bonds formed per kg of oligomer or polymer, moles of remaining OH groups per kg of oligomer or polymer, moles of remaining CO groups per kg of oligomer or polymer, OH number, acid number if CO is a carboxylic acid, percent weight of isocyanate group in products, percent isocyanate in product stripped of any isocyanate monomer, extent of reaction, percent weight of unreacted monomers, number average molecular weight of hard segment, average number of monomers per hard segment, molecular weight of danglers connected to hard segment, and average number of monomers per soft segment. The one or more instructions may cause the at least one processor to: determine at least one expected property associated with the first oligomer or polymer based on the determined at least one characteristic associated with the first oligomer or polymer. The at least one expected property may include at least one of the following: a mechanical testing property, a physical testing property, a thermal testing property, a rheological testing property, a barrier testing property, a weathering and/or chemical resistance testing property, an adhesion testing property, a flammability testing property, an optical testing property, and an electrical testing property.

In some non-limiting embodiments or aspects, simulating the plurality of oligomer or polymer forming reactions may include the one or more instructions causing the at least one processor to: determine an extent of reaction associated with the plurality of simulated oligomer or polymer forming reactions. The at least one reaction rule may include a relative reactivity of at least one functional group type. The at least one reaction rule may include a first functional group type capable of undergoing a reaction with a second functional group type. The reaction recipe may include an initial plurality of reactive molecules and a subsequent plurality of reactive molecules, where simulating the plurality of oligomer or polymer forming reactions may include simulating the oligomer or polymer forming reactions from the initial plurality of reactive molecules based on the at least one reaction rule, where the one or more instructions cause the at least one processor to simulate a plurality of subsequent oligomer or polymer forming reactions from the plurality of subsequent reactive molecules and molecules and/or oligomers and/or polymers formed from the plurality of oligomer or polymer forming reactions based on the at least one reaction rule. The one or more instructions may cause the at least one processor to: generate reaction instructions for forming the first oligomer or polymer. The one or more instructions may cause the at least one processor to: communicate the reaction instructions to a reactor to cause the reactor to initiate preparation of the first oligomer or polymer. Determining the at least one oligomer or polymer structure associated with the first oligomer or polymer may include determining simulated danglers, sols, and elastic links. Determining the at least one expected property may include analyzing the at least one characteristic based on historical data associated with oligomers or polymers. Analyzing the at least one characteristic based on the historical data may include generating the at least one expected property using a machine learning algorithm. The one or more instructions may cause the at least one processor to: store historical simulation data associated with the plurality of simulated oligomers or polymers; receive a recommendation request, where the recommendation request may include at least one target physical property associated with an oligomer or polymer desired to be produced; query the stored historical simulation data; and generate a recommendation response including reaction instructions for forming an oligomer or polymer having the at least one target physical property based on the historical simulation data. Determining the at least one oligomer or polymer structure associated with the first oligomer or polymer may include identifying the at least one oligomer or polymer structure associated with the first oligomer or polymer based on a component searching algorithm.

In some non-limiting embodiments or aspects, the first oligomer or polymer may include a cured thermoset oligomer or polymer. Determining the at least one oligomer or polymer structure associated with the first oligomer or polymer may include the one or more instructions causing the at least one processor to: identify a soft segment of the first oligomer or polymer and/or a hard segment of the first oligomer or polymer; and analyze the soft segment of the first oligomer or polymer and/or the hard segment of the first oligomer or polymer. The one or more instructions may cause the at least one processor to: store historical simulation data associated with the plurality of simulated oligomers or polymers; receive a message from a reactor, the message including at least one property associated with a material being prepared in the reactor; determine at least one reactor adjustment based on the message and the historical simulation data; and communicate a reply message to the reactor to cause the reactor to initiate the reactor adjustment.

Further embodiments or aspects are set forth in the following numbered clauses:

Clause 1: A method for simulating oligomer or polymer growth, comprising: receiving, with at least one processor, a reaction recipe comprising a plurality of reactive molecules; for each reactive molecule of the plurality of reactive molecules, determining, with at least one processor, at least one functional group associated with the reactive molecule; assigning, with at least one processor, a functional group type to each of the functional groups associated with the plurality of reactive molecules; determining, with at least one processor, at least one reaction rule associated with each functional group type; simulating, with at least one processor, a plurality of oligomer or polymer forming reactions from the plurality of reactive molecules based on the at least one reaction rule to form a plurality of simulated oligomers or polymers; and determining, with at least one processor, at least one oligomer or polymer structure associated with a first oligomer or polymer of the plurality of simulated oligomers or polymers.

Clause 2: The method of clause 1, wherein simulating the plurality of oligomer or polymer forming reactions comprises: associating, with at least one processor, at least one functional group associated with the plurality of reactive molecules with at least one other functional group associated with the plurality of reactive molecules.

Clause 3: The method of clause 1 or 2, wherein simulating the plurality of oligomer or polymer forming reactions comprises: generating, with at least one processor, a first list of a plurality of first type reactive functional groups of the reactive functional groups associated with the plurality of reactive molecules and a second list of a plurality of second type reactive functional groups of the reactive functional groups associated with the plurality of reactive molecules; randomizing, with at least one processor, an order of the plurality of first type reactive functional groups in the first list; randomizing, with at least one processor, an order of the plurality of second type reactive functional groups in the second list; associating, with at least one processor, at least one of the first type reactive functional groups from the first list with at least one of the corresponding second reactive functional groups from the second list based on the randomized orders to form at least one simulated bond of the first oligomer or polymer.

Clause 4: The method of any of clauses 1-3, further comprising: generating, with at least one processor, statistical reaction data based on the plurality of oligomer or polymer forming reactions.

Clause 5: The method of any of clauses 1-4, wherein simulating the plurality of oligomer or polymer forming reactions comprises: associating, with at least one processor, at least one pair of the functional groups associated with the plurality of reactive molecules based on the at least one reaction rule to form a bonded pair.

Clause 6: The method of clause 5, further comprising: assigning, with at least one processor, a bonded group identifier associated with the bonded pair.

Clause 7: The method of any of clauses 1-6, wherein simulating the plurality of oligomer or polymer forming reactions comprises: associating, with at least one processor, a first pair of functional groups associated with the plurality of reactive molecules based on the at least one reaction rule to form a first bonded pair; and subsequently associating, with at least one processor, a second pair of functional groups associated with the plurality of reactive molecules based on the at least one reaction rule to form a second bonded pair.

Clause 8: The method of clause 7, wherein simulating the plurality of oligomer or polymer forming reactions further comprises: adjusting, with at least one processor, the at least one reaction rule between associating the first pair of functional groups and associating the second pair of functional groups.

Clause 9: The method of any of clauses 1-8, further comprising: determining, with at least one processor, at least one characteristic associated with the first oligomer or polymer.

Clause 10: The method of clause 9, wherein the at least one characteristic comprises at least one of the following: moles of effective links per kg of oligomer or polymer, moles of effective links per kg of gel component, moles of effective links per kg of core in a gel component, moles of intramolecular rings formed per kg of oligomer or polymer, moles of intermolecular rings formed per kg of gel component, moles of intermolecular rings formed per kg of core of a gel component, crosslink density, moles of crosslink junctions per kg of oligomer or polymer, moles of dangler links per kg of oligomer or polymer, moles of danglers per kg of gel component, percent weight of sol in gelled oligomer or polymer, percent weight of gel in gelled oligomer or polymer, percent weight of danglers, percent weight of core gel, number average molecular weight of elastic links, weight average molecular weight of elastic links, number average molecular weight of danglers, weight average molecular weight of danglers, molecular weight of danglers weighted by its percent of total oligomer or polymer, molecular weight of elastic links weighted by its percent weight of the oligomer or polymer, number average molecular weight, weight average molecular weight, z-average molecular weight, degree of polymerization, dispersity of reaction product, number of ingredient molecules used in simulation, number of monomers used in simulation, number of ring closures formed, number of oligomer molecules formed, ratio of equivalents (CO/OH) of raw materials, number average OH functionality, number average CO functionality, functional-average functionality of CO, weight average functionality of OH, weight-average OH functionality, weight-average CO functionality, average new bonds formed per oligomer, moles of bonds formed per kg of oligomer or polymer, moles of remaining OH groups per kg of oligomer or polymer, moles of remaining CO groups per kg of oligomer or polymer, OH number, acid number if CO is a carboxylic acid, percent weight of isocyanate group in products, percent isocyanate in product stripped of any isocyanate monomer, extent of reaction, percent weight of unreacted monomers, number average molecular weight of hard segment, average number of monomers per hard segment, molecular weight of danglers connected to hard segment, and average number of monomers per soft segment.

Clause 11: The method of clause 9 or 10, further comprising: determining, with at least one processor, at least one expected property associated with the first oligomer or polymer based on the determined at least one characteristic associated with the first oligomer or polymer.

Clause 12: The method of clause 11, wherein the at least one expected property comprises at least one of the following: a mechanical testing property, a physical testing property, a thermal testing property, a rheological testing property, a barrier testing property, a weathering and/or chemical resistance testing property, an adhesion testing property, a flammability testing property, an optical testing property, and an electrical testing property.

Clause 13: The method of any of clauses 1-12, wherein simulating the plurality of oligomer or polymer forming reactions comprises: determining, with at least one processor, an extent of reaction associated with the plurality of simulated oligomer or polymer forming reactions.

Clause 14: The method of any of clauses 1-13, wherein the at least one reaction rule comprises a relative reactivity of at least one functional group type.

Clause 15: The method of any of clauses 1-14, wherein the at least one reaction rule comprises a first functional group type capable of undergoing a reaction with a second functional group type.

Clause 16: The method of any of clauses 1-15, wherein the reaction recipe comprises an initial plurality of reactive molecules and a subsequent plurality of reactive molecules, wherein simulating the plurality of oligomer or polymer forming reactions comprises simulating the oligomer or polymer forming reactions from the initial plurality of reactive molecules based on the at least one reaction rule, wherein the method further comprises simulating, with at least one processor, a plurality of subsequent oligomer or polymer forming reactions from the plurality of subsequent reactive molecules and molecules and/or oligomers and/or polymers formed from the plurality of oligomer or polymer forming reactions based on the at least one reaction rule.

Clause 17: The method of any of clauses 1-16, further comprising: generating, with at least one processor, reaction instructions for forming the first oligomer or polymer.

Clause 18: The method of clause 17, further comprising: communicating, with at least one processor, the reaction instructions to a reactor to cause the reactor to initiate preparation of the first oligomer or polymer.

Clause 19: The method of any of clauses 1-18, wherein determining the at least one oligomer or polymer structure associated with the first oligomer or polymer comprises determining simulated danglers, sols, and elastic links.

Clause 20: The method of any of clauses 11-19, wherein determining the at least one expected property comprises analyzing the at least one characteristic based on historical data associated with oligomers or polymers.

Clause 21: The method of clause 20, wherein analyzing the at least one characteristic based on the historical data comprises generating the at least one expected property using a machine learning algorithm.

Clause 22: The method of any of clauses 1-21, further comprising: storing, with at least one processor, historical simulation data associated with the plurality of simulated oligomers or polymers; receiving, with at least one processor, a recommendation request, wherein the recommendation request comprises at least one target physical property associated with an oligomer or polymer desired to be produced; querying, with at least one processor, the stored historical simulation data; and generating, with at least one processor, a recommendation response comprising reaction instructions for forming an oligomer or polymer having the at least one target physical property based on the historical simulation data.

Clause 23: The method of any of clauses 1-22, wherein determining the at least one oligomer or polymer structure associated with the first oligomer or polymer comprises identifying the at least one oligomer or polymer structure associated with the first oligomer or polymer based on a component searching algorithm.

Clause 24: The method of any of clauses 1-23, wherein the first oligomer or polymer comprises a cured thermoset oligomer or polymer.

Clause 25: The method of any of clauses 1-24, wherein determining the at least one oligomer or polymer structure associated with the first oligomer or polymer comprises at least one of the following: identifying, with at least one processor, a soft segment of the first oligomer or polymer and/or a hard segment of the first oligomer or polymer; and analyzing, with at least one processor, the soft segment of the first oligomer or polymer and/or the hard segment of the first oligomer or polymer.

Clause 26: The method of any of clauses 1-25, further comprising: storing, with at least one processor, historical simulation data associated with the plurality of simulated oligomers or polymers; receiving, with at least one processor, a message from a reactor, the message comprising at least one property associated with a material being prepared in the reactor; determining, with at least one processor, at least one reactor adjustment based on the message and the historical simulation data; and communicating, with at least one processor, a reply message to the reactor to cause the reactor to initiate the reactor adjustment.

Clause 27: A system for simulating oligomer or polymer growth, comprising at least one processor programmed or configured to: receive a reaction recipe comprising a plurality of reactive molecules; for each reactive molecule of the plurality of reactive molecules, determine at least one functional group associated with the reactive molecule; assign a functional group type to each of the functional groups associated with the plurality of reactive molecules; determine at least one reaction rule associated with each functional group type; simulate a plurality of oligomer or polymer forming reactions from the plurality of reactive molecules based on the at least one reaction rule to form a plurality of simulated oligomers or polymers; and determine at least one oligomer or polymer structure associated with a first oligomer or polymer of the plurality of simulated oligomers or polymers.

Clause 28: The system of clause 27, wherein simulating the plurality of oligomer or polymer forming reactions, comprises the at least one processor being programmed or configured to: associate at least one functional group associated with the plurality of reactive molecules with at least one other functional group associated with the plurality of reactive molecules.

Clause 29: The system of clause 27 or 28, wherein simulating the plurality of oligomer or polymer forming reactions, comprises the at least one processor being programmed or configured to: generate a first list of a plurality of first type reactive functional groups of the reactive functional groups associated with the plurality of reactive molecules and a second list of a plurality of second type reactive functional groups of the reactive functional groups associated with the plurality of reactive molecules; randomize an order of the plurality of first type reactive functional groups in the first list; randomize an order of the plurality of second type reactive functional groups in the second list; associate at least one of the first type reactive functional groups from the first list with at least one of the corresponding second reactive functional groups from the second list based on the randomized orders to form at least one simulated bond of the first oligomer or polymer.

Clause 30: The system of any of clauses 27-29, wherein the at least one processor is programmed or configured to: generate statistical reaction data based on the plurality of oligomer or polymer forming reactions.

Clause 31: The system of any of clauses 27-30, wherein simulating the plurality of oligomer or polymer forming reactions, comprises the at least one processor being programmed or configured to: associate at least one pair of the functional groups associated with the plurality of reactive molecules based on the at least one reaction rule to form a bonded pair.

Clause 32: The system of clause 31, wherein the at least one processor is programmed or configured to: assign a bonded group identifier associated with the bonded pair.

Clause 33: The system of any of clauses 27-32, wherein simulating the plurality of oligomer or polymer forming reactions, comprises the at least one processor being programmed or configured to: associate a first pair of functional groups associated with the plurality of reactive molecules based on the at least one reaction rule to form a first bonded pair; and subsequently associate a second pair of functional groups associated with the plurality of reactive molecules based on the at least one reaction rule to form a second bonded pair.

Clause 34: The system of clause 33, wherein simulating the plurality of oligomer or polymer forming reactions, comprises the at least one processor being programmed or configured to: adjust the at least one reaction rule between associating the first pair of functional groups and associating the second pair of functional groups.

Clause 35: The system of any of clauses 27-34, wherein the at least one processor is programmed or configured to: determine at least one characteristic associated with the first oligomer or polymer.

Clause 36: The system of clause 35, wherein the at least one characteristic comprises at least one of the following: moles of effective links per kg of oligomer or polymer, moles of effective links per kg of gel component, moles of effective links per kg of core in a gel component, moles of intramolecular rings formed per kg of oligomer or polymer, moles of intermolecular rings formed per kg of gel component, moles of intermolecular rings formed per kg of core of a gel component, crosslink density, moles of crosslink junctions per kg of oligomer or polymer, moles of dangler links per kg of oligomer or polymer, moles of danglers per kg of gel component, percent weight of sol in gelled oligomer or polymer, percent weight of gel in gelled oligomer or polymer, percent weight of danglers, percent weight of core gel, number average molecular weight of elastic links, weight average molecular weight of elastic links, number average molecular weight of danglers, weight average molecular weight of danglers, molecular weight of danglers weighted by its percent of total oligomer or polymer, molecular weight of elastic links weighted by its percent weight of the oligomer or polymer, number average molecular weight, weight average molecular weight, z-average molecular weight, degree of polymerization, dispersity of reaction product, number of ingredient molecules used in simulation, number of monomers used in simulation, number of ring closures formed, number of oligomer molecules formed, ratio of equivalents (CO/OH) of raw materials, number average OH functionality, number average CO functionality, functional-average functionality of CO, weight average functionality of OH, weight-average OH functionality, weight-average CO functionality, average new bonds formed per oligomer, moles of bonds formed per kg of oligomer or polymer, moles of remaining OH groups per kg of oligomer or polymer, moles of remaining CO groups per kg of oligomer or polymer, OH number, acid number if CO is a carboxylic acid, percent weight of isocyanate group in products, percent isocyanate in product stripped of any isocyanate monomer, extent of reaction, percent weight of unreacted monomers, number average molecular weight of hard segment, average number of monomers per hard segment, molecular weight of danglers connected to hard segment, and average number of monomers per soft segment.

Clause 37: The system of clause 35 or 36, wherein the at least one processor is programmed or configured to: determine at least one expected property associated with the first oligomer or polymer based on the determined at least one characteristic associated with the first oligomer or polymer.

Clause 38: The system of clause 37, wherein the at least one expected property comprises at least one of the following: a mechanical testing property, a physical testing property, a thermal testing property, a rheological testing property, a barrier testing property, a weathering and/or chemical resistance testing property, an adhesion testing property, a flammability testing property, an optical testing property, and an electrical testing property.

Clause 39: The system of any of clauses 27-38, wherein simulating the plurality of oligomer or polymer forming reactions, comprises the at least one processor being programmed or configured to: determine an extent of reaction associated with the plurality of simulated oligomer or polymer forming reactions.

Clause 40: The system of any of clauses 27-39, wherein the at least one reaction rule comprises a relative reactivity of at least one functional group type.

Clause 41: The system of any of clauses 27-40, wherein the at least one reaction rule comprises a first functional group type capable of undergoing a reaction with a second functional group type.

Clause 42: The system of any of clauses 27-41, wherein the reaction recipe comprises an initial plurality of reactive molecules and a subsequent plurality of reactive molecules, wherein simulating the plurality of oligomer or polymer forming reactions comprises simulating the oligomer or polymer forming reactions from the initial plurality of reactive molecules based on the at least one reaction rule, wherein the at least one processor is programmed or configured to simulate a plurality of subsequent oligomer or polymer forming reactions from the plurality of subsequent reactive molecules and molecules and/or oligomers and/or polymers formed from the plurality of oligomer or polymer forming reactions based on the at least one reaction rule.

Clause 43: The system of any of clauses 27-42, wherein the at least one processor is programmed or configured to: generate reaction instructions for forming the first oligomer or polymer.

Clause 44: The system of clause 43, wherein the at least one processor is programmed or configured to: communicate the reaction instructions to a reactor to cause the reactor to initiate preparation of the first oligomer or polymer.

Clause 45: The system of any of clauses 27-44, wherein determining the at least one oligomer or polymer structure associated with the first oligomer or polymer comprises determining simulated danglers, sols, and elastic links.

Clause 46: The system of any of clauses 37-45, wherein determining the at least one expected property comprises analyzing the at least one characteristic based on historical data associated with oligomers or polymers.

Clause 47: The system of clause 46, wherein analyzing the at least one characteristic based on the historical data comprises generating the at least one expected property using a machine learning algorithm.

Clause 48: The system of any of clauses 27-47, wherein the at least one processor is programmed or configured to: store historical simulation data associated with the plurality of simulated oligomers or polymers; receive a recommendation request, wherein the recommendation request comprises at least one target physical property associated with an oligomer or polymer desired to be produced; query the stored historical simulation data; and generate a recommendation response comprising reaction instructions for forming an oligomer or polymer having the at least one target physical property based on the historical simulation data.

Clause 49: The system of any of clauses 27-48, wherein determining the at least one oligomer or polymer structure associated with the first oligomer or polymer comprises identifying the at least one oligomer or polymer structure associated with the first oligomer or polymer based on a component searching algorithm.

Clause 50: The system of any of clauses 27-49, wherein the first oligomer or polymer comprises a cured thermoset oligomer or polymer.

Clause 51: The system of any of clauses 27-50, wherein determining the at least one oligomer or polymer structure associated with the first oligomer or polymer comprises the at least one processor programmed or configured to: identify a soft segment of the first oligomer or polymer and/or a hard segment of the first oligomer or polymer; and analyze the soft segment of the first oligomer or polymer and/or the hard segment of the first oligomer or polymer.

Clause 52: The system of any of clauses 27-51, wherein the at least one processor is programmed or configured to: store historical simulation data associated with the plurality of simulated oligomers or polymers; receive a message from a reactor, the message comprising at least one property associated with a material being prepared in the reactor; determine at least one reactor adjustment based on the message and the historical simulation data; and communicate a reply message to the reactor to cause the reactor to initiate the reactor adjustment.

Clause 53: A computer program product for simulating oligomer or polymer growth comprising at least one non-transitory computer-readable medium including one or more instructions that, when executed by at least one processor, cause the at least one processor to: receive a reaction recipe comprising a plurality of reactive molecules; for each reactive molecule of the plurality of reactive molecules, determine at least one functional group associated with the reactive molecule; assign a functional group type to each of the functional groups associated with the plurality of reactive molecules; determine at least one reaction rule associated with each functional group type; simulate a plurality of oligomer or polymer forming reactions from the plurality of reactive molecules based on the at least one reaction rule to form a plurality of simulated oligomers or polymers; and determine at least one oligomer or polymer structure associated with a first oligomer or polymer of the plurality of simulated oligomers or polymers.

Clause 54: The computer program product of clause 53, wherein simulating the plurality of oligomer or polymer forming reactions comprises the one or more instructions causing the at least one processor to: associate at least one functional group associated with the plurality of reactive molecules with at least one other functional group associated with the plurality of reactive molecules.

Clause 55: The computer program product of clause 53 or 54, wherein simulating the plurality of oligomer or polymer forming reactions comprises the one or more instructions causing the at least one processor to: generate a first list of a plurality of first type reactive functional groups of the reactive functional groups associated with the plurality of reactive molecules and a second list of a plurality of second type reactive functional groups of the reactive functional groups associated with the plurality of reactive molecules; randomize an order of the plurality of first type reactive functional groups in the first list; randomize an order of the plurality of second type reactive functional groups in the second list; associate at least one of the first type reactive functional groups from the first list with at least one of the corresponding second reactive functional groups from the second list based on the randomized orders to form at least one simulated bond of the first oligomer or polymer.

Clause 56: The computer program product of any of clauses 53-55, wherein the one or more instructions cause the at least one processor to: generate statistical reaction data based on the plurality of oligomer or polymer forming reactions.

Clause 57: The computer program product of any of clauses 53-56, wherein simulating the plurality of oligomer or polymer forming reactions comprises the one or more instructions causing the at least one processor to: associate at least one pair of the functional groups associated with the plurality of reactive molecules based on the at least one reaction rule to form a bonded pair.

Clause 58: The computer program product of clause 57, wherein the one or more instructions cause the at least one processor to: assign a bonded group identifier associated with the bonded pair.

Clause 59: The computer program product of any of clauses 53-58, wherein simulating the plurality of oligomer or polymer forming reactions comprises the one or more instructions causing the at least one processor to: associate a first pair of functional groups associated with the plurality of reactive molecules based on the at least one reaction rule to form a first bonded pair; and subsequently associate a second pair of functional groups associated with the plurality of reactive molecules based on the at least one reaction rule to form a second bonded pair.

Clause 60: The computer program product of clause 59, wherein simulating the plurality of oligomer or polymer forming reactions comprises the one or more instructions causing the at least one processor to: adjust the at least one reaction rule between associating the first pair of functional groups and associating the second pair of functional groups.

Clause 61: The computer program product of any of clauses 53-60, wherein the one or more instructions cause the at least one processor to: determine at least one characteristic associated with the first oligomer or polymer.

Clause 62: The computer program product of clause 61, wherein the at least one characteristic comprises at least one of the following: moles of effective links per kg of oligomer or polymer, moles of effective links per kg of gel component, moles of effective links per kg of core in a gel component, moles of intramolecular rings formed per kg of oligomer or polymer, moles of intermolecular rings formed per kg of gel component, moles of intermolecular rings formed per kg of core of a gel component, crosslink density, moles of crosslink junctions per kg of oligomer or polymer, moles of dangler links per kg of oligomer or polymer, moles of danglers per kg of gel component, percent weight of sol in gelled oligomer or polymer, percent weight of gel in gelled oligomer or polymer, percent weight of danglers, percent weight of core gel, number average molecular weight of elastic links, weight average molecular weight of elastic links, number average molecular weight of danglers, weight average molecular weight of danglers, molecular weight of danglers weighted by its percent of total oligomer or polymer, molecular weight of elastic links weighted by its percent weight of the oligomer or polymer, number average molecular weight, weight average molecular weight, z-average molecular weight, degree of polymerization, dispersity of reaction product, number of ingredient molecules used in simulation, number of monomers used in simulation, number of ring closures formed, number of oligomer molecules formed, ratio of equivalents (CO/OH) of raw materials, number average OH functionality, number average CO functionality, functional-average functionality of CO, weight average functionality of OH, weight-average OH functionality, weight-average CO functionality, average new bonds formed per oligomer, moles of bonds formed per kg of oligomer or polymer, moles of remaining OH groups per kg of oligomer or polymer, moles of remaining CO groups per kg of oligomer or polymer, OH number, acid number if CO is a carboxylic acid, percent weight of isocyanate group in products, percent isocyanate in product stripped of any isocyanate monomer, extent of reaction, percent weight of unreacted monomers, number average molecular weight of hard segment, average number of monomers per hard segment, molecular weight of danglers connected to hard segment, and average number of monomers per soft segment.

Clause 63: The computer program product of clause 61 or 62, wherein the one or more instructions cause the at least one processor to: determine at least one expected property associated with the first oligomer or polymer based on the determined at least one characteristic associated with the first oligomer or polymer.

Clause 64: The computer program product of clause 63, wherein the at least one expected property comprises at least one of the following: a mechanical testing property, a physical testing property, a thermal testing property, a rheological testing property, a barrier testing property, a weathering and/or chemical resistance testing property, an adhesion testing property, a flammability testing property, an optical testing property, and an electrical testing property.

Clause 65: The computer program product of any of clauses 53-64, wherein simulating the plurality of oligomer or polymer forming reactions comprises the one or more instructions causing the at least one processor to: determine an extent of reaction associated with the plurality of simulated oligomer or polymer forming reactions.

Clause 66: The computer program product of any of clauses 53-65, wherein the at least one reaction rule comprises a relative reactivity of at least one functional group type.

Clause 67: The computer program product of any of clauses 53-66, wherein the at least one reaction rule comprises a first functional group type capable of undergoing a reaction with a second functional group type.

Clause 68: The computer program product of any of clauses 53-67, wherein the reaction recipe comprises an initial plurality of reactive molecules and a subsequent plurality of reactive molecules, wherein simulating the plurality of oligomer or polymer forming reactions comprises simulating the oligomer or polymer forming reactions from the initial plurality of reactive molecules based on the at least one reaction rule, wherein the one or more instructions cause the at least one processor to simulate a plurality of subsequent oligomer or polymer forming reactions from the plurality of subsequent reactive molecules and molecules and/or oligomers and/or polymers formed from the plurality of oligomer or polymer forming reactions based on the at least one reaction rule.

Clause 69: The computer program product of any of clauses 53-68, wherein the one or more instructions cause the at least one processor to: generate reaction instructions for forming the first oligomer or polymer.

Clause 70: The computer program product of clause 69, wherein the one or more instructions cause the at least one processor to: communicate the reaction instructions to a reactor to cause the reactor to initiate preparation of the first oligomer or polymer.

Clause 71: The computer program product of any of clauses 53-70, wherein determining the at least one oligomer or polymer structure associated with the first oligomer or polymer comprises determining simulated danglers, sols, and elastic links.

Clause 72: The computer program product of any of clauses 63-71, wherein determining the at least one expected property comprises analyzing the at least one characteristic based on historical data associated with oligomers or polymers.

Clause 73: The computer program product of clause 72, wherein analyzing the at least one characteristic based on the historical data comprises generating the at least one expected property using a machine learning algorithm.

Clause 74: The computer program product of any of clauses 53-73, wherein the one or more instructions cause the at least one processor to: store historical simulation data associated with the plurality of simulated oligomers or polymers; receive a recommendation request, wherein the recommendation request comprises at least one target physical property associated with an oligomer or polymer desired to be produced; query the stored historical simulation data; and generate a recommendation response comprising reaction instructions for forming an oligomer or polymer having the at least one target physical property based on the historical simulation data.

Clause 75: The computer program product of any of clauses 53-74, wherein determining the at least one oligomer or polymer structure associated with the first oligomer or polymer comprises identifying the at least one oligomer or polymer structure associated with the first oligomer or polymer based on a component searching algorithm.

Clause 76: The computer program product of any of clauses 53-75, wherein the first oligomer or polymer comprises a cured thermoset oligomer or polymer.

Clause 77: The computer program product of any of clauses 53-76, wherein determining the at least one oligomer or polymer structure associated with the first oligomer or polymer comprises the one or more instructions causing the at least one processor to: identify a soft segment of the first oligomer or polymer and/or a hard segment of the first oligomer or polymer; and analyze the soft segment of the first oligomer or polymer and/or the hard segment of the first oligomer or polymer.

Clause 78: The computer program product of any of clauses 53-77, wherein the one or more instructions cause the at least one processor to: store historical simulation data associated with the plurality of simulated oligomers or polymers; receive a message from a reactor, the message comprising at least one property associated with a material being prepared in the reactor; determine at least one reactor adjustment based on the message and the historical simulation data; and communicate a reply message to the reactor to cause the reactor to initiate the reactor adjustment.

These and other features and characteristics of the present invention, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and the claims, the singular form of “a,” “an,” and “the” include plural referents unless the context clearly dictates otherwise.

### BRIEF DESCRIPTION OF THE DRAWINGS

FIG. 1 shows a reaction recipe according to some non-limiting embodiments or aspects;

FIG. 2 shows a list of molecule and functional group identifiers according to some non-limiting embodiments or aspects;

FIG. 3 shows a list of randomized and associated functional group identifiers according to some non-limiting embodiments or aspects;

FIG. 4 shows a list of bond identifiers associated with simulated bonds formed form the associated functional group identifiers from FIG. 3 according to some non-limiting embodiments or aspects;

FIG. 5 shows a simulated reaction mixture having unreacted monomers according to some non-limiting embodiments or aspects;

FIG. 6 shows the simulated reaction mixture of FIG. 5 having simulated bonds between certain functional groups according to some non-limiting embodiments or aspects;

FIG. 7 shows the simulated reaction mixture of FIG. 6 showing the resulting oligomer/polymer structure formed from the simulation according to some non-limiting embodiments or aspects;

FIG. 8 shows a simulated oligomer or polymer structure according to some non-limiting embodiments or aspects;

FIG. 9 shows generated predictive property models according to some non-limiting embodiments or aspects; and

FIG. 10 shows a system for simulating oligomer or polymer growth according to some non-limiting embodiments or aspects.

### DETAILED DESCRIPTION

For purposes of the description hereinafter, the terms “end,” “upper,” “lower,” “right,” “left,” “vertical,” “horizontal,” “top,” “bottom,” “lateral,” “longitudinal,” and derivatives thereof shall relate to the invention as it is oriented in the drawing figures. However, it is to be understood that the invention may assume various alternative variations and step sequences, except where expressly specified to the contrary. It is also to be understood that the specific devices and processes illustrated in the attached drawings, and described in the following specification, are simply exemplary embodiments or aspects. Hence, specific dimensions and other physical characteristics related to the embodiments or aspects disclosed herein are not to be considered as limiting.

As used herein, the term “application programming interface” (API) may refer to computer code that allows communication between different systems or (hardware and/or software) components of systems. For example, an API may include function calls, functions, subroutines, communication protocols, fields, and/or the like usable and/or accessible by other systems or other (hardware and/or software) components of systems.

As used herein, the terms “communication” and “communicate” may refer to the reception, receipt, transmission, transfer, provision, and/or the like, of information (e.g., data, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection (e.g., a direct communication connection, an indirect communication connection, and/or the like) that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively transmit information to the second unit. As another example, a first unit may be in communication with a second unit if at least one intermediary unit (e.g., a third unit located between the first unit and the second unit) processes information received from the first unit and communicates the processed information to the second unit. In some non-limiting embodiments, a message may refer to a network packet (e.g., a data packet, and/or the like) that includes data. It will be appreciated that numerous other arrangements are possible.

As used herein, the term “computing device” may refer to one or more electronic devices configured to process data. A computing device may, in some examples, include the necessary components to receive, process, and output data, such as a processor, a display, a memory, an input device, a network interface, and/or the like. A computing device may be a mobile device. As an example, a mobile device may include a cellular phone (e.g., a smartphone or standard cellular phone), a portable computer, a wearable device (e.g., watches, glasses, lenses, clothing, and/or the like), a personal digital assistant (PDA), and/or other like devices. A computing device may also be a desktop computer, server, or other form of non-mobile computer.

As used herein, the term “machine learning algorithm” may refer to an algorithm for applying at least one predictive model to a data set. A machine learning algorithm may train at least one predictive model through expansion of the data set by continually or intermittently updating the data set with results of instances of an industrial process. Examples of machine learning algorithms may include supervised and/or unsupervised techniques such as decision trees, gradient boosting, logistic regression, artificial neural networks, Bayesian statistics, learning automata, Hidden Markov Modeling, linear classifiers, quadratic classifiers, association rule learning, or the like. As used herein, the term “machine learning model” may refer to a predictive model at least partially generated by a machine learning algorithm.

As used herein, the term “polymer” refers to a molecule comprising a plurality of repeat units derived from a smaller molecule called a monomer. The term “oligomer” refers to a low-molecular weight polymer.

As used herein, the term “user interface” or “graphical user interface” refers to a generated display, such as one or more graphical user interfaces (GUIs) with which a user may interact, either directly or indirectly (e.g., through a keyboard, mouse, touchscreen, etc.).

Non-limiting embodiments or aspects of the present disclosure are directed to a method, system, and computer program product for simulating oligomer or polymer growth. Non-limiting embodiments or aspects enable a user to simulate, using a computing device, oligomer or polymer growth and determine the simulated oligomer or polymer structure formed and/or a characteristic associated with the oligomer or polymer and/or an expected property associated with the oligomer or polymer. Simulating oligomer or polymer growth may enable a user to determine the expected product (oligomer or polymer formed) associated with a proposed reaction recipe without physically performing the reaction, resulting in a savings in both time and materials. Non-limiting embodiments or aspects may utilize graph theory analysis to determine a structure and/or a characteristic associated with the oligomer or polymer. Non-limiting embodiments or aspects may utilize historical data and/or machine learning algorithms to determine an expected property associated with the oligomer or polymer. Non-limiting embodiments or aspects may simulate oligomer or polymer growth by simultaneously forming all simulated bonds, thus improving the speed and efficiency with which the user may simulate the growth of large oligomers or polymers.

A method for simulating oligomer or polymer growth may include: receiving, with at least one processor, a reaction recipe comprising a plurality of reactive molecules; for each reactive molecule of the plurality of reactive molecules, determining, with at least one processor, at least one functional group associated with the reactive molecule; assigning, with at least one processor, a functional group type to each of the functional groups associated with the plurality of reactive molecules; determining, with at least one processor, at least one reaction rule associated with each functional group type; simulating, with at least one processor, a plurality of oligomer or polymer forming reactions from the plurality of reactive molecules based on the at least one reaction rule to form a plurality of simulated oligomers or polymers; and determining, with at least one processor, at least one oligomer or polymer structure associated with a first oligomer or polymer of the plurality of simulated oligomers or polymers.

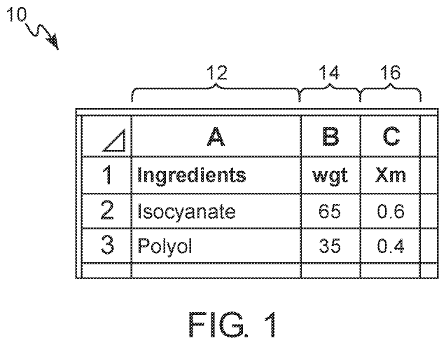

Referring to FIG. 1, a reaction recipe 10 is shown for simulating oligomer or polymer growth. The oligomer or polymer to be simulated may include a thermoplastic polymer, a thermoset polymer, or some combination thereof. The reaction recipe 10 may specify a plurality of reactive molecules 12 to be included in the simulated reaction. The example reaction recipe 10 shown in FIG. 1 includes two reactive molecules 12 (an isocyanate and a polyol); however the reaction recipe 10 may include more than two reactive molecules 12. The reaction recipe 10 may further include non-reactive components, such as additives, catalysts, and the like, which do not necessarily react to form the oligomer or polymer. The reaction recipe 10 may further include an amount 14 associated with each component (e.g., reactive molecules 12) in the reaction recipe 10. The amount may include a weight percent, a mole percent, a weight fraction, a mole fraction (e.g., Xm 16 shown in FIG. 1), a weight, moles, and/or the like associated with each component of the reaction recipe 10.

The reaction recipe 10 may further include process instructions associated with simulating oligomer or polymer growth. For example, the process instructions may include an order in which steps are to be performed, a temperature for the simulated reaction, a time at which to simulate agitation of the reaction mixture, a setting associated with the stirring, and the like.

The reaction recipe 10 may further include a characteristic associated with each component. Characteristics associated with the component may include, but are not limited to, a molecular weight associated with the component, a functionality associated with each component (e.g., number of free hydroxyl groups, number of free acid groups, number of free isocyanate groups, and the like), a relative reactivity of each component, characteristics from which molecular weight and functionality can be derived, and the like.

In some non-limiting embodiments or aspects, the simulated oligomer or polymer growth may include all components in the reaction recipe 10 being included in the simulated reaction mixture at the same time to effect the simulated reaction. In other non-limiting embodiments or aspects, the simulated oligomer or polymer growth may include certain components in the reaction recipe being initially included in the simulated reaction mixture and reacted together and subsequently adding further reactive molecules 12 to continue the simulated reaction (e.g., a multi-step reaction). The reaction recipe 10 may indicate such a scenario by including an order of addition associated with each component in the reaction recipe 10.

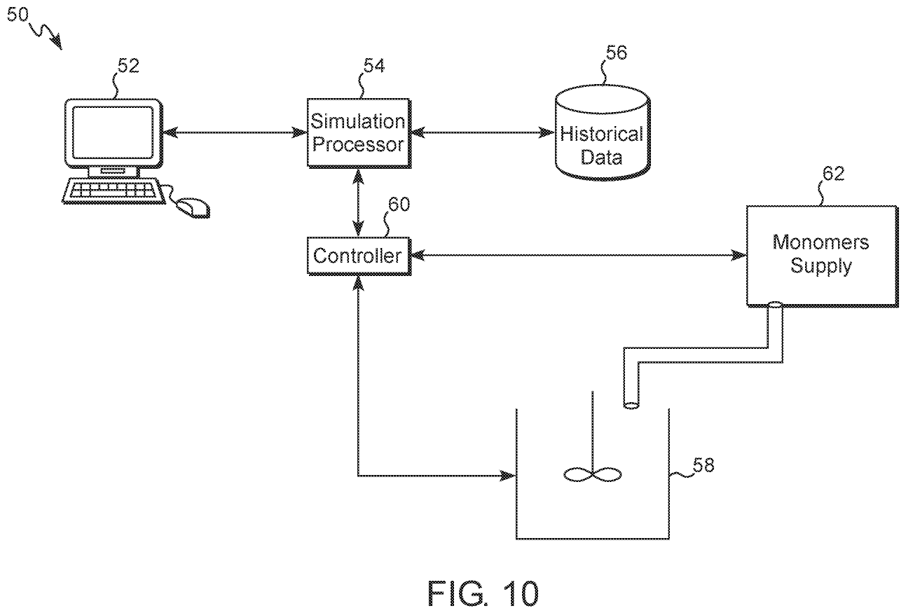

Referring to FIG. 10, a system 50 is shown for simulating oligomer or polymer growth. The system 50 may include a computing device 52 associated with a user. The computing device 52 may communicate with a simulation processor 54 configured to simulate oligomer or polymer growth. The simulation processor 54 may communicate with a historical data database 56 for storing historical data associated with prior simulations and for storing data associated with known products (e.g., oligomers or polymers), such as characteristics and properties associated with known products. The simulation processor 54 may communicate with a controller 60 configured to control a reactor 58 and a monomer supply, which may be in communication with the reactor 58 to add monomers and other components to the reactor 58.

The computing device 52 may communicate the reaction recipe 10 to the simulation processor 54 to initiate simulation of oligomer or polymer growth. The user may specify the reaction recipe 10 by inputting data into or specifying data on a user interface displayed on the computing device 52.

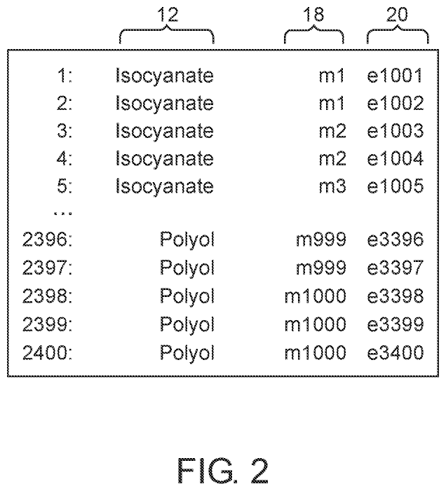

Referring to FIGS. 2 and 10, the simulation processor 54 may, in response to receiving the reaction recipe 10, determine a number of molecules and/or a number of each type (e.g., the isocyanate and the polyol in FIG. 2) of molecule associated with the simulation based on the reaction recipe 10. The number molecules may be determined by the amount 14 of each component specified in the reaction recipe 10. Each molecule may be assigned a unique molecule identifier 18 by the simulation processor 54.

With continued reference to FIGS. 2 and 10, the simulation processor 54 may, in response to receiving the reaction recipe 10, determine at least one functional group associated with each of the reactive molecules 12. For example, the simulation processor 54 may analyze the reactive molecules 12 of the reaction recipe 10 to determine the functional groups associated with each of the reactive molecules 12. For example, the simulation processor 54 may determine the functional groups associated with each of the reactive molecules 12 based on data included in the reaction recipe 10 which specifies the functional groups associated with the reactive molecules 12. The simulation processor 54 may assign a functional group type to each functional group on each of the reactive molecules 12 (e.g., functional group types include a hydroxyl group, an acid group, an isocyanate group, and the like). The simulation processor 54 may assign a unique functional group identifier 20 to each functional group of the reactive molecules 12.

Referring to FIG. 2, a list is shown based on the reaction recipe from FIG. 1. The list includes a list of every functional group included in the reaction recipe, sorted based on the reactive molecules 12. At the top of the list, isocyanate reactive molecules 12 are listed. Because the particular isocyanate used in this non-limiting example has two isocyanate functional groups per molecule, the same isocyanate molecule is listed in two separate rows (ml in rows 1 and 2 of the list are associated with the same isocyanate molecule because they have the same molecule identifier 18). Each of the two lines for the isocyanate, however, have a different functional group identifier 20, as each of the two isocyanate functional groups of the isocyanate reactive molecule 12 receives a different functional group identifier 20. At the bottom of the list, polyol reactive molecules 12 are listed. Because the particular polyol used in this non-limiting example has three functional groups per molecule, the same polyol molecules is listed in three separate rows (m1000 in rows 2398-2400 of the list are associated with the same polyol molecule because they have the same molecule identifier 18). Each of the three lines for the polyol, however, have a different functional group identifier 20, as each of the three hydroxyl functional groups of the polyol reactive molecule 12 receives a different functional group identifier 20.

In this way, for any reaction recipe 10 received, the simulation processor 54 may identify each molecule and functional group thereof in the simulated reaction, such that the structure of the simulated oligomer or polymer may be determined after the simulated reaction.

The simulation processor 54 may determine at least one reaction rule associated with each of the reactive molecules and/or the functional group types. The at least one reaction rule may specify which functional groups types may react with other functional groups, such as specifying that isocyanate groups may react with hydroxyl groups in the example shown in FIG. 2. The at least one reaction rule may specify which functional groups types cannot react, such as specifying that isocyanate groups cannot react with other isocyanate groups and that hydroxyl groups cannot react with other hydroxyl groups in example shown in FIG. 2. The reaction rule may specify a relative reactivity of at least one functional group type compared to another functional group type, one reactive molecule 12 compared to another reactive molecule 12, of one functional group within a reactive molecule 12 compared to another functional group within the same reactive molecule 12, and the like. The simulation processor 54 may use these reaction rules during the simulation to determine which bonds are more likely to form in the simulated reaction.

The simulation processor 54 may determine the reaction rules based on data included in the reaction recipe, data from the historical data database 56, or other data received by or programmed on the simulation processor 54 associated with the reactivity of reactive molecules 12 and/or functional groups.

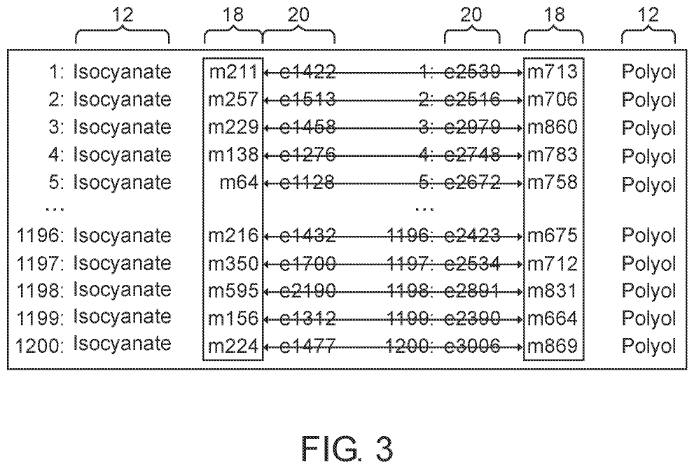

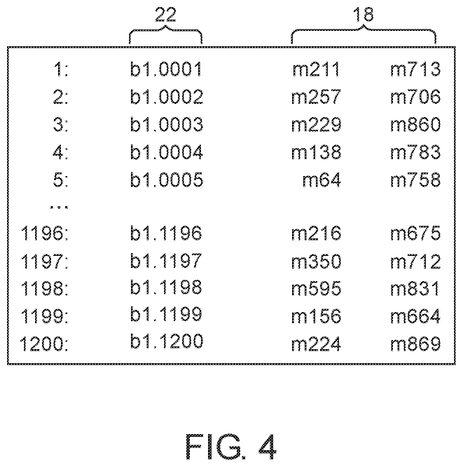

Referring to FIGS. 3-4, the simulation processor 54 may simulate a plurality of oligomer or polymer forming reactions for the reactive molecules 12 based on the reaction rules to form a plurality of simulated oligomers or polymers. The simulation processor 54 may execute at least one Monte Carlo-type simulation to determine the reactions between functional groups that occur to form at least one oligomer or polymer. A plurality of simulations may be run to determine the different potential oligomers or polymers that may be formed from the reaction recipe 10 and the likelihood that an oligomer or polymer having a specific structure or characteristic may be formed.

Simulated bonds may be formed during the simulated reaction by associating at least one functional group of a reactive molecule 12 with at least one functional group of another reactive molecule 12 to form a bonded pair. The association may occur in one or more data structures. The simulated bonds may be formed based on the reaction rules. As shown in FIG. 4, a bonded pair may be assigned a unique bond identifier 22 to identify the bonded pair. For example, as shown in FIG. 4, row 1, monomer m211 and monomer m713 (functional groups thereof) were simulated to form a bonded pair having a bond identifier 22 of b1.0001. The specific functional groups having a functional group identifier 20 may also be identified as the functional groups of the monomers which form the simulated bond (see FIG. 3).

Referring again to FIG. 3, simulating the oligomer or polymer forming reactions may include generating a first list of a plurality of a first type of reactive functional groups associated with the reactive molecules 12 (see the list of isocyanate functional groups on the left of the list) and a second list of a plurality of a second type of reactive functional groups associated with the reactive molecules 12 (see the list of hydroxyl functional groups on the right of the list). The order of the first list may be randomized such that the molecule identifiers 18 and the functional group identifiers 20 are not necessarily in numerical order. The order of the second list may be randomized such that the molecule identifiers 18 and the functional group identifiers 20 are not necessarily in numerical order. At least one of the first type reactive functional groups from the first list and at least one of the corresponding second reactive functional groups from the second list, based on the randomized orders, may be associated to form at least one simulated bond of the first oligomer or polymer. This association is shown in FIG. 3 by the arrow matching an isocyanate monomer in a row of the table with a polyol monomer in that same row. The simulation processor 54 may associate the corresponding functional groups from the first and second list simultaneously (e.g., concurrently or substantially concurrently), such that all simulated bonds are formed substantially at the same time and/or in the same step of a simulated reaction.

In some non-limiting embodiments or aspects, the simulation processor 54 may not simultaneously form all simulated bonds at the same time. The simulation processor 54 may associate a first pair of functional groups associated with the plurality of reactive molecules 12 based on the at least one reaction rule to form a first bonded pair. Subsequently, the simulation processor may associate a second pair of functional groups associated with the plurality of reactive molecules 12 based on the at least one reaction rule to form a second bonded pair. Between the formation of the first bonded pair and the second bonded pair, at least one of the reaction rules may be adjusted. For example, the reaction rules may be adjusted to reflect a change in relative reactivity of the monomers and/or functional groups based on a previously simulated bond being formed (e.g., a functional group on a monomer being less reactive because another functional group on that same monomer already formed a simulated bond).

In some non-limiting embodiments or aspects, the reaction to be simulated may include a plurality of reactive steps (e.g., a multi-step reaction). The reaction recipe 10 may include an initial plurality of reactive molecules and a subsequent plurality of reactive molecules. Simulating the plurality of oligomer or polymer forming reactions may include simulating the oligomer or polymer forming reactions from the initial plurality of reactive molecules based on the at least one reaction rule. Subsequently, the simulation processor 54 may simulate a plurality of subsequent oligomer or polymer forming reactions from the plurality of subsequent reactive molecules and molecules and/or oligomers and/or polymers formed from the plurality of oligomer or polymer forming reactions (the initial reactions) based on the at least one reaction rule.

In some non-limiting embodiments or aspects, the simulation may run until all functional groups have been reacted and/or the reaction rules dictate that no further functional groups are capable of undergoing a reaction (e.g., an excess amount of a reactive component is included such that there are no functional groups with which it can react). In this way, the simulation may run until the extent of reaction of the simulated reaction is 100%. In some non-limiting embodiments or aspects, the simulation may run to an extent of reaction less than 100%. The extent of reaction to which the simulation is to run may be specified in the reaction recipe 10. The simulation processor 54 may determine the extent of reaction to which the reaction is to run.

Based on the simulated reaction performed by the simulation processor 54, the simulation processor 54 may determine at least one oligomer or polymer structure associated with an oligomer or polymer formed from the plurality of simulated oligomers or polymers. As previously mentioned, a plurality of simulations may be run to determine the different oligomer or polymer structures that may be formed and the likelihood that certain structures are formed. Statistical reaction data may be generated based on this plurality of simulations. The statistical reaction data may be analyzed to determine the likelihood that certain oligomer or polymer structures are to occur.

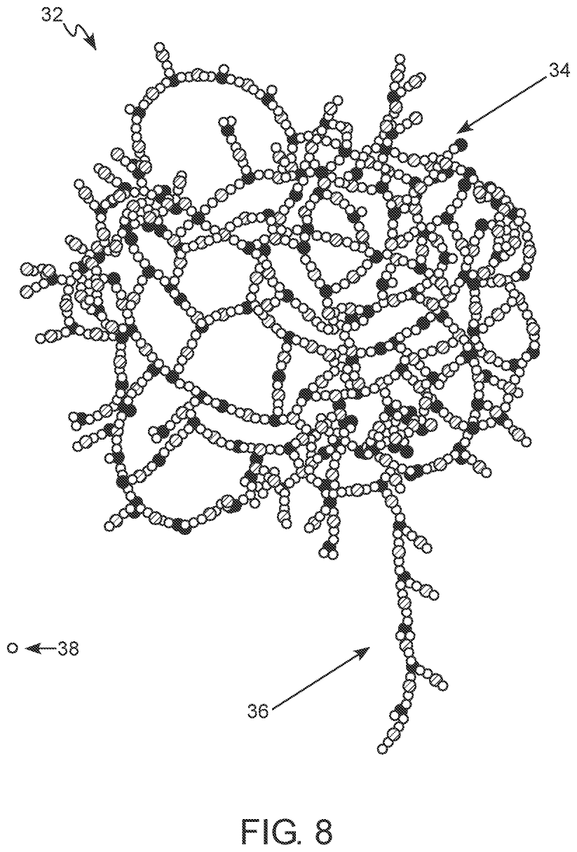

Determining the oligomer or polymer structure may include determining simulated danglers, sols, and elastic links in the simulated oligomer or polymer. FIG. 8 shows a non-limiting example of a simulated structure 32 of a simulated oligomer or polymer. The simulated structure 32 may include a gel region 34, which includes elastic links to make up the gel region. The simulated structure 32 may include danglers 36, which are pendant groups attached to the gel region 34. The simulated structure 32 may include a sol 38, which is an oligomer not connected to the gel region 34. Each of these regions may be identified based on the structure formed by the simulation.

The simulated structure 32 of the oligomer or polymer formed during the simulation may be generated by a program using graph theory analysis, such as igraph or NetworkX. The graph theory analysis may result in the simulated structure 32 being display on the computing device 52, such that the user can view the simulated structure 32. The graph theory analysis may include a connected component searching algorithm.

For a simulated oligomer or polymer, at least one characteristic associated with the oligomer or polymer may be determined based on the determined structure thereof. As used herein, the term “characteristic” refers to a feature of the oligomer or polymer which can be directly determined from the simulated structure of the oligomer or polymer itself. Non-limiting examples of characteristics include, but are not limited to: moles of effective links per kg of oligomer or polymer, moles of effective links per kg of gel component, moles of effective links per kg of core in a gel component, moles of intramolecular rings formed per kg of oligomer or polymer, moles of intermolecular rings formed per kg of gel component, moles of intermolecular rings formed per kg of core of a gel component, crosslink density, moles of crosslink junctions per kg of oligomer or polymer, moles of dangler links per kg of oligomer or polymer, moles of danglers per kg of gel component, percent weight of sol in gelled oligomer or polymer, percent weight of gel in gelled oligomer or polymer, percent weight of danglers, percent weight of core gel, number average molecular weight of elastic links, weight average molecular weight of elastic links, number average molecular weight of danglers, weight average molecular weight of danglers, molecular weight of danglers weighted by its percent of total oligomer or polymer, molecular weight of elastic links weighted by its percent weight of the oligomer or polymer, number average molecular weight, weight average molecular weight, z-average molecular weight, degree of polymerization, dispersity of reaction product, number of ingredient molecules used in simulation, number of monomers used in simulation, number of ring closures formed, number of oligomer molecules formed, ratio of equivalents (CO/OH) of raw materials, number average OH functionality, number average CO functionality, functional-average functionality of CO, weight average functionality of OH, weight-average OH functionality, weight-average CO functionality, average new bonds formed per oligomer, moles of bonds formed per kg of oligomer or polymer, moles of remaining OH groups per kg of oligomer or polymer, moles of remaining CO groups per kg of oligomer or polymer, OH number, acid number if CO is a carboxylic acid, percent weight of isocyanate group in products, percent isocyanate in product stripped of any isocyanate monomer, extent of reaction, percent weight of unreacted monomers, number average molecular weight of hard segment, average number of monomers per hard segment, molecular weight of danglers connected to hard segment, and average number of monomers per soft segment.

In some non-limiting embodiment or aspects, determining the oligomer or polymer structure may include identifying a soft segment of the oligomer or polymer and/or a hard segment of the oligomer or polymer. The hard or soft segment of the oligomer or polymer may be determined by the composition of the oligomer or polymer in a particular region of the oligomer or polymer. For example, in a simulated polyurethane, a soft segment may be identified based on the presence of polyether or polyester polyol in the region of the oligomer or polymer, while the presence of a diisocyanate or chain extender in the region of the oligomer or polymer may be identified as a hard segment. In this way, identifying the bonds formed in the various regions of the simulated structure may be used to determine whether the region of the oligomer or polymer is a hard segment and/or a soft segment. The simulation processor 54 may analyze the soft segment and/or the hard segment of the oligomer or polymer. Analyzing the hard and/or soft segment may include determining a number average molecular weight of hard and/or soft segment, average number of monomers per hard and/or soft segment, molecular weight of danglers connected to hard and/or soft segment, and the like.

For a simulated oligomer or polymer, at least one expected property associated with the oligomer or polymer may be determined based on at least one of the determined characteristics of the oligomer or polymer. As used herein, the term “property” refers to a feature of the oligomer or polymer which cannot be directly determined from the simulated structure of the oligomer or polymer itself but is a feature exhibited by the oligomer or polymer and can be measured using at least one test method. Non-limiting examples of properties include, but are not limited to: a mechanical testing property (e.g., tensile strength, compressional strength, flexural strength, torsional strength, impact strength, % elongation, modulus, shore hardness (shore A, shore D), and the like), a physical testing property (density, crystallinity, and the like), a thermal testing property (melting point, glass transition temperature (Tg), thermal conductivity, and the like), a rheological testing property (viscosity and the like), a barrier testing property (permeance and the like), a weathering and/or chemical resistance testing property (UV degradation and the like), an adhesion testing property (work of adhesion and the like), a flammability testing property (limiting oxygen index, Underwriters Laboratory (UL94) testing (e.g., flammability rating), and the like), an optical testing property (gloss, transparency, clarity, haze, color, surface aspect, refractive index, and the like), and an electrical testing property (electrical conductivity and the like).

The simulation processor 54 may determine an expected property of a simulated oligomer or polymer based at least partially on at least one of the characteristics determined for the oligomer or polymer. The expected property may be determined based on an analysis of at least one characteristic of the oligomer or polymer based on historical data associated with known oligomers or polymers stored in the historical data database 56. The properties of the known oligomers or polymers stored in the historical data database 56 may have been determined by measuring the property thereof.

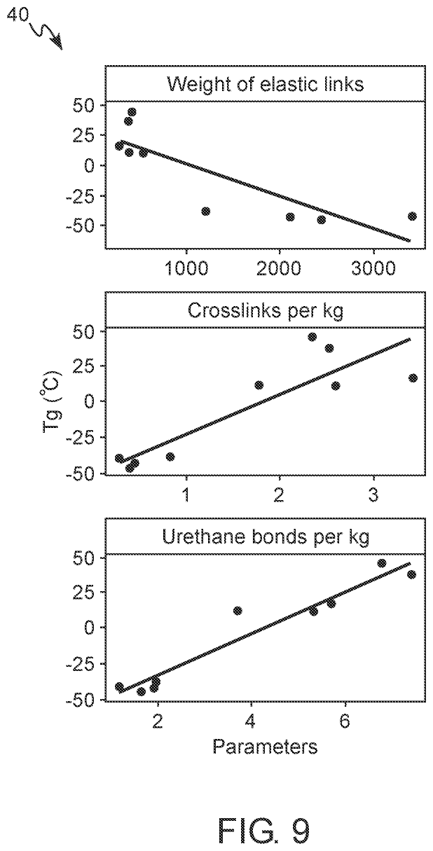

As shown in FIG. 9, a property predictive model 40 may be generated based on data from the historical data database 56. The property predictive model 40 may include a relationship between a characteristic of known oligomers or polymers and a property of the known oligomers or polymers. For example, as shown in FIG. 9, property predictive models 40 illustrate a relationship between a property of known oligomers or polymers (Tg) and characteristics (weight of elastic links, crosslinks per kg, urethane bonds per kg) associated with the oligomers or polymers. A fit equation may be determined from the property predictive model 40 which may allow an expected property to be determined based on a characteristic of any oligomer or polymer, including the simulated oligomers or polymers. A regression analysis may be performed to determine how well the fit equation models the property based on the determined characteristic. The expected property of the simulated oligomer or polymer may be determined based on at least one characteristic of that oligomer or polymer and how the property predictive model 40 associates that characteristic with the property of interest. Machine learning techniques may also be used to determine the expected property of the oligomer or polymer by applying a suitable machine learning algorithm to the historical data in the historical data database 56 based on at least one characteristic of the oligomer or polymer and may be used to generate the property predictive model 40. The machine learning algorithm may utilize, as inputs, one or more characteristics in addition to the historical data to output an expected property. Examples of Machine Learning algorithm include, but are not limited to, Random Forest, SVM, xgBoost, elasticNet, neurol networks, Gaussian Process, and multivariate linear regression.

Referring to FIG. 10, the simulation processor 54 may store historical simulation data associated with the simulated oligomers or polymers in the historical data database 56. The historical simulation data may include data associated with the reaction recipe 10, the generated statistical reaction data, the determined structure of the simulated oligomers or polymers, the characteristics associated with the simulated oligomers or polymers, the expected properties associated with the oligomers or polymers, and the like.

With continued reference to FIG. 10, the simulation processor 54 may generate reaction instructions for forming the simulated oligomer or polymer. The reaction instructions may include at least a portion of the reaction recipe 10. The reaction instructions may be communicated to the controller 60 configured to control the monomers supply 62 and/or the reactor 58. The controller 60 may determine the monomers from the monomer supply 62 to be added to the reactor 58, the time at which the monomers are added to the reactor 58, the rate at which the monomers are added to the reactor, and the like. The controller 60 may control, the rate at which agitation in the reactor 58 occurs, the temperature of the reactor 58, the flow rate at the outlet of the reactor 58, and the like. The reaction instructions may cause the reactor 58 to prepare the oligomer or polymer.

With continued reference to FIG. 10, in some non-limiting embodiments or aspects, the computing device 52 may communicate a recommendation request to the simulation processor 54. The recommendation request may include at least one target physical property associated with an oligomer or polymer desired to be produced. The simulation processor 54 may communicate with the historical data database 56 to query the stored historical simulation data. Based on the query, the simulation processor 54 may generate a recommendation response and communicate the recommendation response to the computing device 52. The recommendation response may include a proposed reaction recipe, a recommend oligomer or polymer structure, and/or reaction instructions for forming an oligomer or polymer having the at least one target physical property based on the historical simulation data. Upon the user selecting one of the recommended oligomers or polymers, the simulation processor 54 may communicate with the controller 60 to cause the selected oligomer or polymer to be produced by the reactor 58.

In some non-limiting embodiments or aspects, during preparation of an oligomer or polymer by the reactor 58, the reactor 58 may communicate a message to the simulation processor 54, and the message may include at least one property associated with a material being prepared in the reactor 58. Based on the property of the material as included in the message, the simulation processor 54 may determine at least one reactor adjustment based on the historical simulation data included in the historical data database 56. The simulation processor 54 may utilize a machine learning algorithm to determine the reactor adjustment, such as based on effective adjustments made for historically prepared oligomers or polymers. The simulation processor 54 may communicate a reply message to the reactor 58 to cause the reactor 58 to initiate the reactor adjustment.

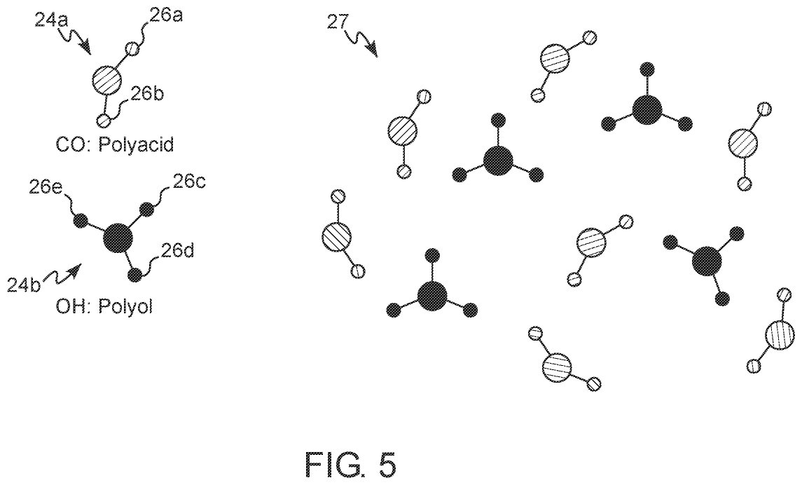

Referring to FIGS. 5-7, a representation of a simulation of oligomer or polymer growth is shown according to some non-limiting embodiments or aspects. The non-limiting example shows polymer growth associated with a reaction between a polyacid having a plurality of acid functional groups and a polyol having hydroxyl functional groups. The polyacid in the non-limiting example comprise a diacid (e.g., adipic acid), but it will be appreciated that other acids are possible, and other monomers containing different functional groups (other than acid functional groups) are possible. The polyol in the non-limiting example comprise a triol (e.g., trimethylolpropane), but it will be appreciated that other polyols are possible, and other monomers containing different functional groups (other than hydroxyl functional groups) are possible.

With continued reference to FIGS. 5-7, a plurality of monomers 24a, 24b are shown. Monomer 24a is a polyacid having two acid functional groups 26a, 26b. Monomer 24b is a polyol having three hydroxyl functional groups 26c, 26d, 26e.

Referring to FIG. 5, a reaction mixture 27 including a plurality of the monomers 24a, 24b is shown, and the monomers 24a, 24b are unbonded to one another, representing the reaction mixture 27 before the simulated bonding occurs.

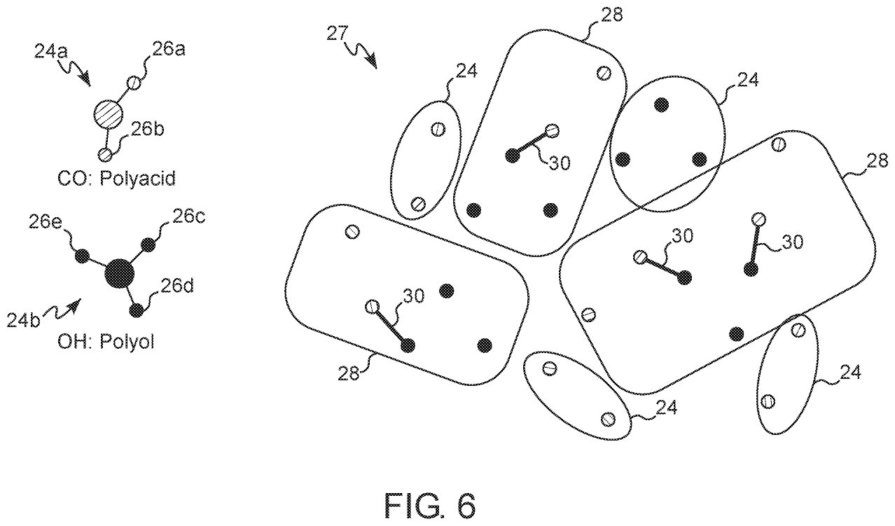

Referring to FIG. 6, the reaction mixture 27 is shown with only the functional groups 26a-26e of the monomers 24a, 24b shown. The simulated bonding has at least partially occurred, as certain of the functional groups are connected to one another via a bond 30. According to the reactions rules in this example, an acid functional group is only capable of forming a bond with a hydroxyl functional group, and a hydroxyl functional group is only capable of forming a bond with an acid functional group. Based on the simulated bonding, the monomers 24 may be identified, and the formed oligomers or polymers 28 may be identified.

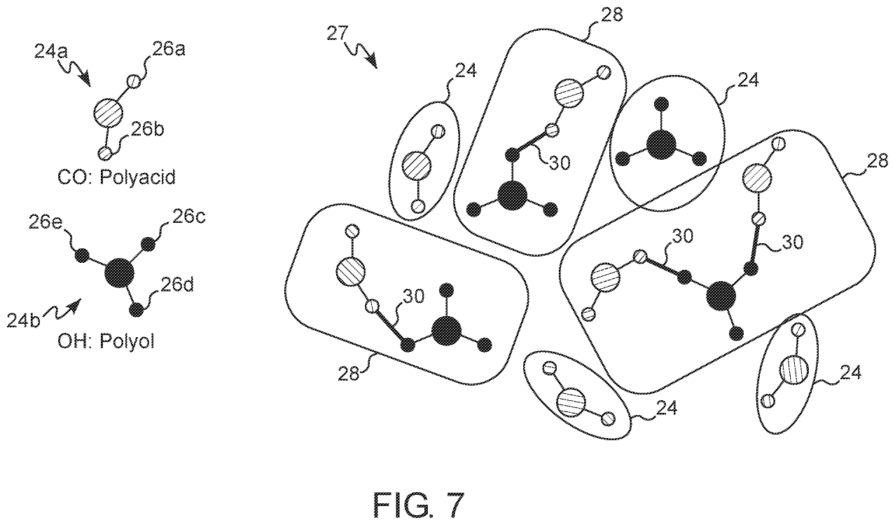

Referring to FIG. 7, the reaction mixture 27 is identical to the reaction mixture 27 from FIG. 6 except the entirety of the molecules (not just the functional groups) included in the reaction mixture 27 are shown. The simulated structure of the monomers 24 and of the oligomers or polymers 28 can be determined based on this graphical representation.

In a further, non-limiting embodiment or aspect, a computer program product for simulating oligomer or polymer growth includes at least one non-transitory computer readable medium including program instructions that, when executed by at least one processor, cause the at least one processor to execute one of the previously-described methods. The at least one processor may include the simulation processor.

### SIMULATION EXAMPLES

The following example is provided to illustrate embodiments of the system, method, and computer program product for simulating oligomer or polymer growth and is not meant to be limiting.

### Example 1

### One Step Reaction

### Receive the Reaction Recipe

A reaction recipe as shown in Table 1 is received by the simulation processor for simulating the growth of an oligomer or polymer:

TABLE 1

| | | | | | Relative | Order |
|---|---|---|---|---|---|---|
| | | | | | Reactiv- | of Addi- |
| Ingredient | Mw | mol | f_OH | f_NCO | ity | tion |
| Polyethylene | 1000 | 1 | 2 | 0 | 1:1 | 1 |
| glycol (PEG | | | | | | |
| 1000) | | | | | | |
| 1,6-Hexanediol | 118 | 1 | 2 | 0 | 1:1 | 1 |
| Toluene di- | 172 | 4 | 0 | 2 | 18:1 | 1 |
| isocyanate (TDI) | | | | | | |

According to the reaction recipe, all ingredients are added simultaneously, based on the order of addition of each component being the same. The relative reactivity of the two isocyanate groups in the TDI is different, with one of the isocyanate groups of the TDI being 18× more reactive than the other isocyanate group of the TDI. All hydroxyl groups of the polyols have the same relative reactivity. In this example, 12 monomers are simulated, but in other simulations over 100,000 monomers may be simulated.

### Generate the List of Functional Groups

From the reaction recipe and the number of monomers, the reaction processor generates a list of all the functional groups in the simulation. The eqId column in Table 2 associates a functional group identifier with each functional group (isocyanate group or hydroxyl group). The generated list of functional groups is shown in Table 2 below.

TABLE 2

| | | | | | Relative |
|---|---|---|---|---|---|
| eqId | monomerId | Ingredient | n_OH | n_NCO | Reactivity |
| e01 | m01 | 1,6-Hexanediol | 1 | 0 | 1 |
| e02 | m01 | 1,6-Hexanediol | 1 | 0 | 1 |
| e03 | m02 | 1,6-Hexanediol | 1 | 0 | 1 |
| e04 | m02 | 1,6-Hexanediol | 1 | 0 | 1 |
| e05 | m03 | PEG 1000 | 1 | 0 | 1 |
| e06 | m03 | PEG 1000 | 1 | 0 | 1 |
| e07 | m04 | PEG 1000 | 1 | 0 | 1 |
| e08 | m04 | PEG 1000 | 1 | 0 | 1 |
| e09 | m05 | TDI | 0 | 1 | 18 |
| e10 | m05 | TDI | 0 | 1 | 1 |
| e11 | m06 | TDI | 0 | 1 | 18 |
| e12 | m06 | TDI | 0 | 1 | 1 |
| e13 | m07 | TDI | 0 | 1 | 18 |
| e14 | m07 | TDI | 0 | 1 | 1 |
| e15 | m08 | TDI | 0 | 1 | 18 |
| e16 | m08 | TDI | 0 | 1 | 1 |
| e17 | m09 | TDI | 0 | 1 | 18 |
| e18 | m09 | TDI | 0 | 1 | 1 |
| e19 | m10 | TDI | 0 | 1 | 18 |
| e20 | m10 | TDI | 0 | 1 | 1 |
| e21 | m11 | TDI | 0 | 1 | 18 |
| e22 | m11 | TDI | 0 | 1 | 1 |
| e23 | m12 | TDI | 0 | 1 | 18 |
| e24 | m12 | TDI | 0 | 1 | 1 |

Split the List into Functional Group Types

To link the hydroxyl functional groups with the isocyanate functional groups (simulate bonding), the simulation processor splits the functional group list, column eqId from Table 2, into two: (1) —OH functional groups: [e01, e02, e03, e04, e05, e06, e07, e08]; (2) —NCO functional groups [e09, e10, e11, e12, e13, e14, e15, e16, e17, e18, e19, e20, e21, e22, e23, e24]. In this example, the list of hydroxyl groups has a length of eight and the list of isocyanate functional groups has a length of sixteen

### Randomize the Functional Group Lists

Both lists are randomized, with the randomization determined probabilistically by the weight specified in the Relative Reactivity column. Upon randomizing the two lists, the order was generated to be: (1) —OH functional groups: [e06, e01, e04, e07, e03, e05, e02, e08]; (2) —NCO functional groups: [e13, e09, e21, e111, e19, e17, e23, e15, e12, e18, e20, e14, e22, e10, e24, e16].

### Form all Simulated Bonds

In this example, the bonds are formed in one step, simultaneously, by joining the two lists, such that the first hydroxyl functional group is bonded to the first isocyanate group, and so on. Each bond formed is assigned a unique identifier, as shown in Tables 3 and 4 below as chembondId. Two cases of forming the simulated bonds, with Case 1 having an extent of reaction of 100% and Case 2 having an extent of reaction of 87.5%, are shown.

### Case 1

Table 3 shows the reaction proceeding to 100% completion. The unmatched NCO functional groups are those that remain unreacted due to stoichiometry.

TABLE 3

| | | | Reaction |
|---|---|---|---|
| OH_groups | NCO_groups | chembondId | Step |
| e06 | e13 | b1 | 1 |
| e01 | e09 | b2 | 1 |
| e04 | e21 | b3 | 1 |
| e07 | e11 | b4 | 1 |
| e03 | e19 | b5 | 1 |
| e05 | e17 | b6 | 1 |
| e02 | e23 | b7 | 1 |
| e08 | e15 | b8 | 1 |
| — | e12 | — | — |
| — | e18 | — | — |
| — | e20 | — | — |
| — | e14 | — | — |
| — | e22 | — | — |
| — | e10 | — | — |
| — | e24 | — | — |
| — | e16 | — | — |

### Case 2

Table 4 shows the reaction proceeding to 87.5% completion. In this case, one of the seven OH functional groups does not form a bond due to the lower extent of reaction.

TABLE 4

| | | | Reaction |
|---|---|---|---|
| OH_groups | NCO_groups | chembondId | Step |
| e06 | e13 | b1 | 1 |
| e01 | e09 | b2 | 1 |
| e04 | e21 | b3 | 1 |
| e07 | e11 | b4 | 1 |
| e03 | e19 | b5 | 1 |
| e05 | e17 | b6 | 1 |
| e02 | e23 | b7 | 1 |
| e08 | e15 | — | — |
| — | e12 | — | — |
| — | e18 | — | — |
| — | e20 | — | — |
| — | e14 | — | — |
| — | e22 | — | — |
| — | e10 | — | — |
| — | e24 | — | — |
| — | e16 | — | — |

### Determine Simulated Structure

To determine the simulated structure of the resulting prepolymer, the simulation processor reframes the lists from Tables 2 and 3 as a graph object using graph theory. Graph theory is a branch of mathematics which studies nodes (vertices) and the links (edges) that connect them. Using this technique, an adjacency list is generated as shown in Table 5. In this example, nodes and links are defined in the following ways: (1) functional groups that are chemically bonded are nodes, and the chemical bond is the link; and (2) functional groups as identified with eqId in Table 2, and the monomer it belongs to, monomerId in Table 2, are the nodes. That the functional group belongs to the monomer is the link.

TABLE 5

| node_a | node_b |
|---|---|
| e06 | e13 |
| e01 | e09 |
| e04 | e21 |
| e07 | e11 |
| e03 | e19 |
| e05 | e17 |
| e02 | e23 |
| e08 | e15 |
| m01 | e01 |
| m01 | e02 |
| m02 | e03 |
| m02 | e04 |
| m03 | e05 |
| m03 | e06 |
| m04 | e07 |
| m04 | e08 |
| m05 | e09 |
| m05 | e10 |
| m06 | e11 |
| m06 | e12 |
| m07 | e13 |
| m07 | e14 |
| m08 | e15 |
| m08 | e16 |
| m09 | e17 |
| m09 | e18 |
| m10 | e19 |
| m10 | e20 |
| m11 | e21 |
| m11 | e22 |
| m12 | e23 |
| m12 | e24 |

A graph theory analysis package, such as igraph, can be used to convert the adjacency list from Table 5 into a graph object defining the full simulated structure.

### Example 2

### Multi-Step Reaction

### Receive the Reaction Recipe

A reaction recipe as shown in Table 6 is received by the simulation processor for simulating the growth of an oligomer or polymer. In this example, the simulated reaction proceeds in two steps, which is indicated by the Order of Addition column. In this example, 14 monomers are simulated:

TABLE 6

| | | | | | | | Order |
|---|---|---|---|---|---|---|---|
| | | | | | Relative | | of |
| | | | | | Reactiv- | Step | Addi- |
| Ingredient | Mw | mol | f_OH | f_NCO | ity | Added | tion |
| PEG 1000 | 1000 | 1 | 2 | 0 | 1:1 | 1 | 1 |
| 1,6- | 118 | 1 | 2 | 0 | 1:1 | 1 | 1 |
| Hexanediol | | | | | | | |
| TDI | 172 | 4 | 0 | 2 | 18:1 | 1 | 1 |
| PEG 1000 | 1000 | 1 | 2 | 0 | 1:1 | 2 | 2 |

### Generate the List of Functional Groups

From the reaction recipe and the number of monomers, the reaction processor generates a list of all the functional groups in the simulation. The generated list of functional groups is shown in Table 7 below.

TABLE 7

| | | | | | Order | |
|---|---|---|---|---|---|---|
| | | | | | of | Relative |
| | mono- | | | | Addi- | Reactiv- |
| eqId | merId | Ingredient | n_OH | n_NCO | tion | ity |
| e01 | m01 | 1,6-Hexanediol | 1 | 0 | 1 | 1 |
| e02 | m01 | 1,6-Hexanediol | 1 | 0 | 1 | 1 |
| e03 | m02 | 1,6-Hexanediol | 1 | 0 | 1 | 1 |
| e04 | m02 | 1,6-Hexanediol | 1 | 0 | 1 | 1 |
| e05 | m03 | PEG 1000 | 1 | 0 | 1 | 1 |
| e06 | m03 | PEG 1000 | 1 | 0 | 1 | 1 |
| e07 | m04 | PEG 1000 | 1 | 0 | 1 | 1 |
| e08 | m04 | PEG 1000 | 1 | 0 | 1 | 1 |
| e09 | m05 | TDI | 0 | 1 | 1 | 18 |
| e10 | m05 | TDI | 0 | 1 | 1 | 1 |
| e11 | m06 | TDI | 0 | 1 | 1 | 18 |
| e12 | m06 | TDI | 0 | 1 | 1 | 1 |

TABLE 7
(Continued)

| | | | | | Order of | Relative |
|---|---|---|---|---|---|---|
| eqId | monomerId | Ingredient | n_OH | n_NCO | Addition | Reactivity |
| e13 | m07 | TDI | 0 | 1 | 1 | 18 |
| e14 | m07 | TDI | 0 | 1 | 1 | 1 |
| e15 | m08 | TDI | 0 | 1 | 1 | 18 |
| e16 | m08 | TDI | 0 | 1 | 1 | 1 |
| e17 | m09 | TDI | 0 | 1 | 1 | 18 |
| e18 | m09 | TDI | 0 | 1 | 1 | 1 |
| e19 | m10 | TDI | 0 | 1 | 1 | 18 |
| e20 | m10 | TDI | 0 | 1 | 1 | 1 |
| e21 | m11 | TDI | 0 | 1 | 1 | 18 |
| e22 | m11 | TDI | 0 | 1 | 1 | 1 |
| e23 | m12 | TDI | 0 | 1 | 1 | 18 |
| e24 | m12 | TDI | 0 | 1 | 1 | 1 |
| e25 | m13 | PEG 1000 | 1 | 0 | 2 | 1 |
| e26 | m13 | PEG 1000 | 1 | 0 | 2 | 1 |
| e27 | m14 | PEG 1000 | 1 | 0 | 2 | 1 |
| e28 | m14 | PEG 1000 | 1 | 0 | 2 | 1 |

Split the List into Functional Group Types

For the first reaction step, the simulation processor considers only those functional groups that were added in the first step (Order of Addition column=1). The simulation processor filters out those rows and splits the column eqId into two lists according to functional group type: (1) —OH functional groups: [e01, e02, e03, e04, e05, e06, e07, e08]; (2) —NCO functional groups [e09, e10, e11, e12, e13, e14, e15, e16, e17, e18, e19, e20, e21, e22, e23, e24]. In this example, the list of hydroxyl groups has a length of eight and the list of isocyanate functional groups has a length of sixteen

### Randomize the Functional Group Lists

Both lists are randomized, with the randomization determined probabilistically by the weight specified in the Relative Reactivity column. Upon randomizing the two lists, the order was generated to be: (1) —OH functional groups: [e06, e01, e04, e07, e03, e05, e02, e08]; (2) —NCO functional groups. [e13, e09, e21, e11, e19, e17, e23, e15, e12, e18, e20, e14, e22, e10, e24, e16].

### Form all Simulated Bonds in Step 1

In this example, the bonds in step 1 of the reaction are formed in one step, simultaneously, by joining the two lists, such that the first hydroxyl functional group is bonded to the first isocyanate group, and so on. Each bond formed is assigned a unique identifier, as shown in Table 8 below as chembondIDd. The extent of reaction in this example is 100%.

Table 8 shows simulated bonds resulting from step 1 of the reaction. The unmatched NCO functional groups are those that remain unreacted due to stoichiometry.

TABLE 8

| | | | Reaction |
|---|---|---|---|
| OH_groups | NCO_groups | chembondId | Step |
| e06 | e13 | b1 | 1 |
| e01 | e09 | b2 | 1 |
| e04 | e21 | b3 | 1 |
| e07 | e11 | b4 | 1 |
| e03 | e19 | b5 | 1 |
| e05 | e17 | b6 | 1 |
| e02 | e23 | b7 | 1 |
| e08 | e15 | b8 | 1 |
| — | e12 | — | — |
| — | e18 | — | — |
| — | e20 | — | — |
| — | e14 | — | — |
| — | e22 | — | — |
| — | e10 | — | — |
| — | e24 | — | — |
| — | e16 | — | — |

### Form all Simulated Bonds in Step 2

After step 1 of the reaction, no hydroxyl groups remain unreacted, and eight isocyanate groups remain unreacted, as the step 1 of the reaction ran to 100% completion. The polyol (PEG 1000) is added in step 2 of the reaction, as indicated in Tables 6 and 7. Thus, the hydroxyl groups e25-e28 are added to the reaction. These hydroxyl groups are first randomized probabilistically by the weight specified in the Relative Reactivity column. Upon randomizing the list of hydroxyl functional groups added in step 2, the order was generated to be: (3) —OH functional groups: [e25, e28, e27, e26]

Table 9 shows simulated bonds resulting from step 2 of the reaction. The unmatched NCO functional groups are those that remain unreacted due to stoichiometry.

TABLE 9

| | | | Reaction |
|---|---|---|---|
| OH_groups | NCO_groups | chembondId | Step |
| e06 | e13 | b1 | 1 |
| e01 | e09 | b2 | 1 |
| e04 | e21 | b3 | 1 |
| e07 | e11 | b4 | 1 |
| e03 | e19 | b5 | 1 |
| e05 | e17 | b6 | 1 |
| e02 | e23 | b7 | 1 |
| e08 | e15 | b8 | 1 |
| e25 | e12 | b9 | 2 |
| e28 | e18 | b10 | 2 |
| e27 | e20 | b11 | 2 |
| e26 | e14 | b12 | 2 |
| — | e22 | — | — |
| — | e10 | — | — |
| — | e24 | — | — |
| — | e16 | — | — |

### Determine Simulated Structure

The simulated structure for the oligomer or polymer formed in Example 2 may be determined using the same procedure as described in connection with Example 1.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A method for simulating oligomer or polymer growth, comprising:
receiving, with at least one processor, a reaction recipe comprising a plurality of reactive molecules;
for each reactive molecule of the plurality of reactive molecules, determining, with at least one processor, at least one functional group associated with the reactive molecule;
assigning, with at least one processor, a functional group type to each of the functional groups associated with the plurality of reactive molecules;
determining, with at least one processor, at least one reaction rule associated with each functional group type;
simulating, with at least one processor, a plurality of oligomer or polymer forming reactions from the plurality of reactive molecules based on the at least one reaction rule to form a plurality of simulated oligomers or polymers; and
determining, with at least one processor, at least one oligomer or polymer structure associated with a first oligomer or polymer of the plurality of simulated oligomers or polymers.

2. The method of claim 1, wherein simulating the plurality of oligomer or polymer forming reactions comprises:
associating, with at least one processor, at least one functional group associated with the plurality of reactive molecules with at least one other functional group associated with the plurality of reactive molecules.

3. The method of claim 1, wherein simulating the plurality of oligomer or polymer forming reactions comprises:
generating, with at least one processor, a first list of a plurality of first type reactive functional groups of the reactive functional groups associated with the plurality of reactive molecules and a second list of a plurality of second type reactive functional groups of the reactive functional groups associated with the plurality of reactive molecules;
randomizing, with at least one processor, an order of the plurality of first type reactive functional groups in the first list;
randomizing, with at least one processor, an order of the plurality of second type reactive functional groups in the second list;
associating, with at least one processor, at least one of the first type reactive functional groups from the first list with at least one of the corresponding second reactive functional groups from the second list based on the randomized orders to form at least one simulated bond of the first oligomer or polymer.

4. The method of claim 1, further comprising:
generating, with at least one processor, statistical reaction data based on the plurality of oligomer or polymer forming reactions.

5. The method of claim 1, wherein simulating the plurality of oligomer or polymer forming reactions comprises:
associating, with at least one processor, at least one pair of the functional groups associated with the plurality of reactive molecules based on the at least one reaction rule to form a bonded pair.

6. The method of claim 5, further comprising:
assigning, with at least one processor, a bonded group identifier associated with the bonded pair.

7. The method of claim 1, wherein simulating the plurality of oligomer or polymer forming reactions comprises:
associating, with at least one processor, a first pair of functional groups associated with the plurality of reactive molecules based on the at least one reaction rule to form a first bonded pair; and
subsequently associating, with at least one processor, a second pair of functional groups associated with the plurality of reactive molecules based on the at least one reaction rule to form a second bonded pair.

8. The method of claim 7, wherein simulating the plurality of oligomer or polymer forming reactions further comprises:
adjusting, with at least one processor, the at least one reaction rule between associating the first pair of functional groups and associating the second pair of functional groups.

9. The method of claim 1, further comprising:
determining, with at least one processor, at least one characteristic associated with the first oligomer or polymer.

10. The method of claim 9, wherein the at least one characteristic comprises at least one of the following: moles of effective links per kg of oligomer or polymer, moles of effective links per kg of gel component, moles of effective links per kg of core in a gel component, moles of intramolecular rings formed per kg of oligomer or polymer, moles of intermolecular rings formed per kg of gel component, moles of intermolecular rings formed per kg of core of a gel component, crosslink density, moles of crosslink junctions per kg of oligomer or polymer, moles of dangler links per kg of oligomer or polymer, moles of danglers per kg of gel component, percent weight of sol in gelled oligomer or polymer, percent weight of gel in gelled oligomer or polymer, percent weight of danglers, percent weight of core gel, number average molecular weight of elastic links, weight average molecular weight of elastic links, number average molecular weight of danglers, weight average molecular weight of danglers, molecular weight of danglers weighted by its percent of total oligomer or polymer, molecular weight of elastic links weighted by its percent weight of the oligomer or polymer, number average molecular weight, weight average molecular weight, z-average molecular weight, degree of polymerization, dispersity of reaction product, number of ingredient molecules used in simulation, number of monomers used in simulation, number of ring closures formed, number of oligomer molecules formed, ratio of equivalents (CO/OH) of raw materials, number average OH functionality, number average CO functionality, functional-average functionality of CO, weight average functionality of OH, weight-average OH functionality, weight-average CO functionality, average new bonds formed per oligomer, moles of bonds formed per kg of oligomer or polymer, moles of remaining OH groups per kg of oligomer or polymer, moles of remaining CO groups per kg of oligomer or polymer, OH number, acid number if CO is a carboxylic acid, percent weight of isocyanate group in products, percent isocyanate in product stripped of any isocyanate monomer, extent of reaction, percent weight of unreacted monomers, number average molecular weight of hard segment, average number of monomers per hard segment, molecular weight of danglers connected to hard segment, and average number of monomers per soft segment.

11. The method of claim 9, further comprising:
determining, with at least one processor, at least one expected property associated with the first oligomer or polymer based on the determined at least one characteristic associated with the first oligomer or polymer.

12. The method of claim 11, wherein the at least one expected property comprises at least one of the following: a mechanical testing property, a physical testing property, a thermal testing property, a rheological testing property, a barrier testing property, a weathering and/or chemical resistance testing property, an adhesion testing property, a flammability testing property, an optical testing property, and an electrical testing property.

13. The method of claim 1, wherein simulating the plurality of oligomer or polymer forming reactions comprises:
determining, with at least one processor, an extent of reaction associated with the plurality of simulated oligomer or polymer forming reactions.

14. The method of claim 1, wherein the at least one reaction rule comprises a relative reactivity of at least one functional group type.

15. The method of claim 1, wherein the at least one reaction rule comprises a first functional group type capable of undergoing a reaction with a second functional group type.

16. The method of claim 1, wherein the reaction recipe comprises an initial plurality of reactive molecules and a subsequent plurality of reactive molecules,
wherein simulating the plurality of oligomer or polymer forming reactions comprises simulating the oligomer or polymer forming reactions from the initial plurality of reactive molecules based on the at least one reaction rule,
wherein the method further comprises simulating, with at least one processor, a plurality of subsequent oligomer or polymer forming reactions from the plurality of subsequent reactive molecules and molecules and/or oligomers and/or polymers formed from the plurality of oligomer or polymer forming reactions based on the at least one reaction rule.

17. The method of claim 1, further comprising:
generating, with at least one processor, reaction instructions for forming the first oligomer or polymer.

18. The method of claim 17, further comprising:
communicating, with at least one processor, the reaction instructions to a reactor to cause the reactor to initiate preparation of the first oligomer or polymer.

19. The method of claim 1, wherein determining the at least one oligomer or polymer structure associated with the first oligomer or polymer comprises determining simulated danglers, sols, and elastic links.

20. The method of claim 11, wherein determining the at least one expected property comprises analyzing the at least one characteristic based on historical data associated with oligomers or polymers.

21.-28. (canceled)
